(19) United States
(12) Patent Application Publication
(10) Pub. No.: US 2023/0089715 A1
(43) Pub. Date: Mar. 23, 2023

(54) MASITINIB FOR THE TREATMENT OF A MULTIPLE SCLEROSIS PATIENT SUBPOPULATION

(71) Applicant: AB SCIENCE, Paris (FR)

(72) Inventors: Alain MOUSSY, Paris (FR); Colin MANSFIELD, Caen (FR)

(73) Assignee: AB SCIENCE, Paris (FR)

(21) Appl. No.: 17/795,076

(22) PCT Filed: Feb. 19, 2021
(86) PCT No.: PCT/EP2021/054164

§ 371 Date: Jul. 25, 2022

(30) Foreign Application Priority Data
Feb. 20, 2020 (EP) 20305163.6

(51) Int. Cl. A61K 31/496; A61K 38/22; A61K 39/395; A61K 31/52; A61K 31/4188; A61K 31/7076; A61K 31/675; A61K 38/13; A61K 31/137; A61K 31/519; A61K 31/397; A61K 38/21; A61P 25/28

(52) U.S. Cl. CPC A61K 31/496; A61K 38/22; A61K 39/3955; A61K 31/52; A61K 31/4188; A61K 31/7076; A61K 31/675; A61K 38/13; A61K 31/137; A61K 31/519; A61K 31/397; A61K 38/215; A61P 25/28

(57) ABSTRACT

Masitinib, or a pharmaceutically acceptable salt or solvate thereof, for use in the treatment of progressive multiple sclerosis (MS) in a patient who has a time from diagnosis to treatment initiation with masitinib, or the pharmaceutically acceptable salt or solvate thereof, greater than 2 years and/or has a time from onset to treatment initiation with masitinib, or the pharmaceutically acceptable salt or solvate thereof, greater than 5 years. In particular, masitinib, or the pharmaceutically acceptable salt or solvate thereof, is for use in the treatment of primary progressive multiple sclerosis (PPMS) or non-active secondary progressive multiple sclerosis (non-active SPMS) in a patient who has a time from diagnosis to treatment initiation with masitinib, or the pharmaceutically acceptable salt or solvate thereof, greater than 2 years and/or has a time from onset to treatment initiation with masitinib, or the pharmaceutically acceptable salt or solvate thereof, greater than 5 years.

### FIELD OF INVENTION

The present invention relates to the treatment of multiple sclerosis. In particular, the present invention relates to the treatment of progressive multiple sclerosis.

### BACKGROUND OF INVENTION

Multiple sclerosis (MS) is an inflammatory demyelinating disease of the central nervous system (CNS). Multiple sclerosis is characterized by a loss of myelin leading to lesions in the CNS (also sometimes called plaques), an excess number of astroglial cells, varying degrees of axonal pathology, and progressive neurological dysfunction. Said neurological dysfunction may notably manifest as movement disorders (such as decreased muscle strength), sensory disturbances (such as a change in the sensation of hot and cold, touch, tingling), balance disorders, visual disturbances and urinary problems. Diagnosed on average around the age of 30 years (20-40 years), with a female preponderance, multiple sclerosis is the leading cause of nontraumatic neurological disability in young and middle-age adults, and affects about 2.5 million people worldwide. Multiple sclerosis probably results from an interaction of genetic susceptibility with one or more environmental factor(s) including infectious agents such as viruses, the role of which is yet to be proven. In particular, in some patients, multiple sclerosis may result from an abnormal immune response to one or several myelin antigen(s) after exposure to one or more environmental factor(s).

Currently, multiple sclerosis (MS) is considered to encompass four types (also sometimes called disease courses or phenotypes) as defined by the International Advisory Committee on Clinical Trials of MS in 2013. The four types of MS are: clinically isolated syndrome (CIS), relapsing-remitting multiple sclerosis (RRMS or RR-MS), secondary progressive multiple sclerosis (SPMS or SP-MS), and primary progressive multiple sclerosis (PPMS or PP-MS). Clinically isolated syndrome (CIS) is defined as a first episode of neurologic symptoms caused by inflammation and demyelination in the central nervous system, which may or may not be followed by the development of MS. Relapsing-remitting multiple sclerosis (RRMS) is the most common type of MS, occurring in about 85% of patients with MS, and characterized by clearly defined episodes of new or worsening symptoms (i.e., relapses), followed by periods of partial or complete recovery (i.e., remissions). RRMS usually evolves over time to become secondary progressive multiple sclerosis (SPMS), characterized by a worsening of symptoms and/or accumulation of disability over time, with (active SPMS) or without (non-active or relapse-free SPMS) the occurrence of relapses and remissions. It is thus estimated that about 50% of patients with RRMS will develop SPMS within 10 years, and about 90% will develop SPMS within 25 years. Of note, less than 40% of patients continue to suffer from relapses (i.e., clearly defined episodes of new or worsening symptoms) after they transition from RRMS to SPMS. Finally, about 10 to 15% patients with MS suffer from primary progressive multiple sclerosis (PPMS), characterized by a gradual worsening of symptoms and/or accumulation of disability from the onset of the disease, without relapses or remissions.

From the currently defined types of MS, two broad categories can be distinguished: MS presenting with relapses followed by remissions, mainly linked to inflammation; and MS presenting with a gradual progression, without relapses and remissions, mainly linked to nerve degeneration. As used herein, progressive multiple sclerosis (progressive MS) refers to the progressive, non-relapsing, forms of multiple sclerosis and accounts for about 60% of MS patients.

There is currently no cure for multiple sclerosis. The most frequently prescribed treatments aim at managing symptoms and, in RRMS, at managing relapses and bringing about remission. Disease-modifying therapies (DMTs) are also available, aiming at reducing the activity and progression of MS. DMTs thus affect the underlying cause of the disease instead of merely alleviating symptoms of the disease. However, many of the DMTs carry significant health risks and are associated with severe side-effects. Most of the disease-modifying therapies (DMTs) currently available target RRMS. Indeed, DMTs (such as, for example, interferon beta (INF-β), glatiramer acetate, dimethyl fumarate or natalizumab) work primarily by reducing inflammation in the central nervous system. Such DMTs are not suitable for patients with progressive MS characterized by nerve degeneration rather than inflammation. For primary progressive MS, there is only one FDA-approved disease-modifying therapy, ocrelizumab. Patients who receive ocrelizumab are slightly less likely to progress (i.e., to worsen) than those who are untreated. For secondary progressive MS, DMTs are only available for active secondary progressive MS.

Therefore, there is still a need for treatments of progressive multiple sclerosis. In particular, there is a need for treatments able to slow or reverse the gradual worsening of symptoms and/or accumulation of disability occurring over time in progressive multiple sclerosis, and in particular in PPMS and in non-active SPMS.

The present invention thus relates to a 2-aminoarylthiazole derivative, in particular masitinib, or a pharmaceutically acceptable salt or solvate thereof, for use in the treatment of progressive MS in a patient in need thereof, wherein said patient has a time from diagnosis to treatment initiation with the 2-aminoarylthiazole derivative, in particular masitinib, or a pharmaceutically acceptable salt or solvate thereof, greater than 1 year, preferably greater than 2 years, and/or has a time from onset to treatment initiation with the 2-aminoarylthiazole derivative, in particular masitinib, or a pharmaceutically acceptable salt or solvate thereof, greater than 2 years, preferably greater than 5 years.

### SUMMARY

The present invention relates to a 2-aminoarylthiazole derivative, in particular masitinib, or a pharmaceutically acceptable salt or solvate thereof, for use in the treatment of progressive multiple sclerosis (MS) in a patient in need thereof, wherein said patient has a time from diagnosis to treatment initiation with the 2-aminoarylthiazole derivative, in particular masitinib, or a pharmaceutically acceptable salt or solvate thereof, greater than 2 years and/or has a time from onset to treatment initiation with the 2-aminoarylthiazole derivative, in particular masitinib, or a pharmaceutically acceptable salt or solvate thereof, greater than 5 years.

In one embodiment, said patient has a time from diagnosis to treatment initiation with the 2-aminoarylthiazole derivative, in particular masitinib, or a pharmaceutically acceptable salt or solvate thereof, greater than 2 years. In one embodiment, said patient has a time from diagnosis to treatment initiation with the 2-aminoarylthiazole derivative, in particular masitinib, or a pharmaceutically acceptable salt or solvate thereof, greater than 3 years.

In one embodiment, said patient has a time from onset to treatment initiation with the 2-aminoarylthiazole derivative, in particular masitinib, or a pharmaceutically acceptable salt or solvate thereof, greater than 5 years.

In one embodiment, said patient has a time from diagnosis to treatment initiation with the 2-aminoarylthiazole derivative, in particular masitinib, or a pharmaceutically acceptable salt or solvate thereof, greater than 2 years and has a time from onset to treatment initiation with the 2-aminoarylthiazole derivative, in particular masitinib, or a pharmaceutically acceptable salt or solvate thereof, greater than 5 years.

In one embodiment, progressive MS is primary progressive multiple sclerosis (PPMS). In one embodiment, progressive MS is non-active secondary progressive multiple sclerosis (non-active SPMS or nSPMS).

In one embodiment, the 2-aminoarylthiazole derivative has the formula (II):

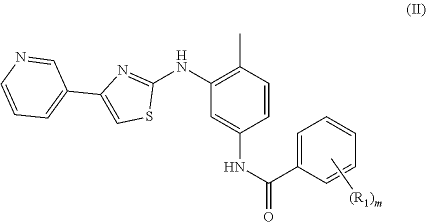

wherein:
R₁ is selected independently from hydrogen, halogen, (C₁-C₁₀) alkyl, (C₃-C₁₀) cycloalkyl group, trifluoromethyl, alkoxy, amino, alkylamino, dialkylamino, a solubilizing group, and (C₁-C₁₀) alkyl substituted by a solubilizing group; and
m is 0-5.

In one preferred embodiment, the 2-aminoarylthiazole derivative, or a pharmaceutically acceptable salt or solvate thereof, is masitinib or a pharmaceutically acceptable salt or solvate thereof. In one embodiment, the pharmaceutically acceptable salt of masitinib is masitinib mesilate.

In one embodiment, the 2-aminoarylthiazole derivative, in particular masitinib, or a pharmaceutically acceptable salt or solvate thereof, is for oral administration. In one embodiment, the 2-aminoarylthiazole derivative, in particular masitinib, or a pharmaceutically acceptable salt or solvate thereof, is for administration at a dose ranging from about 1 mg/kg/day to about 12 mg/kg/day, preferably at a dose of about 4.5 mg/kg/day. In one embodiment, the 2-aminoarylthiazole derivative, in particular masitinib, or a pharmaceutically acceptable salt or solvate thereof, is for administration at an initial dose of about 4.5 mg/kg/day during at least 12 weeks, and then at a dose of about 6 mg/kg/day thereafter, with each dose escalation being subjected to toxicity controls.

In one embodiment, the 2-aminoarylthiazole derivative, in particular masitinib, or a pharmaceutically acceptable salt or solvate thereof, is for administration as a second-line treatment. In one embodiment, said patient failed to respond to a previously administered first-line treatment for multiple sclerosis or showed irreversible progression of the disease despite previous administration of a first-line treatment for multiple sclerosis.

In one embodiment, the 2-aminoarylthiazole derivative, in particular masitinib, or a pharmaceutically acceptable salt or solvate thereof, is for administration with at least one further pharmaceutically active agent. In one embodiment, the at least one further pharmaceutically active agent is selected from the group consisting of adrenocorticotropic hormone (ACTH), alemtuzumab, azathioprine, high-dose biotin (MD1003), cladribine, cyclophosphamide, cyclosporine, dalfampridine, dimethyl fumarate, diroximel fumarate, evobrutinib, fampridine, fingolimod, glatiramer acetate, ibudilast, immunoglobulins, interferon such as interferon beta 1b, interferon beta-1a, or peginterferon beta-1a, laquinimod, methotrexate, mitoxantrone, mycophenolate mofetil, natalizumab, ocrelizumab, ofatumumab, ozanimod, ponesimod, simvastatin, siponimod, teriflunomide, and ublituximab.

Definitions

In the present invention, the following terms have the following meanings:
“About” preceding a figure encompasses plus or minus 10%, or less, of the value of said figure. It is to be understood that the value to which the term “about” refers is itself also specifically, and preferably, disclosed.
“Baseline” as used herein refers to the time preceding the start of the treatment with the 2-aminoarylthiazole derivative, in particular masitinib, or a pharmaceutically acceptable salt or solvate thereof, as described herein. For example, for a given patient, the score on the Expanded Disability Status Scale (or EDSS score) at baseline is the EDSS score prior to the administration to the patient of a 2-aminoarylthiazole derivative, in particular masitinib, or a pharmaceutically acceptable salt or solvate thereof, as described herein.
“Best supportive care” is commonly used to refer to the supportive care given to improve the quality of life of patients who have a serious or life-threatening disease. Thus, as used herein, the terms “best supportive care” and “supportive care” can be used interchangeably. With regards to MS, and in particular progressive MS, supportive care may involve a range of therapies and strategies that can help with managing symptoms and enhancing the quality of life. Examples include medications to help relieve symptoms (in particular medications to help relieve symptoms without acting on the underlying cause), therapies to help enhance strength, mood, and mobility; and lifestyle adjustments that can support physical and mental wellness.
“Palliative care” refers to the active and total care of pain and psychological, spiritual and social problems of patients with a disease that does not respond to curative treatments. Palliative care aims to improve the quality of life of both patients and families.
“Patient” refers to a mammal, preferably a human. According to the present invention, a patient is a mammal, preferably a human, suffering from multiple sclerosis, in particular progressive multiple sclerosis. In one embodiment, the patient is awaiting the receipt of, or is receiving medical care or was/is/will be the object of a medical procedure, or is monitored for the development of multiple sclerosis, in particular progressive multiple sclerosis.
“Pharmaceutically acceptable excipient” or “pharmaceutically acceptable carrier” refers to an excipient or carrier that does not produce any adverse, allergic or other untoward reaction when administered to a mammal, preferably a human. It includes any and all solvents, such as, for example, dispersion media, coatings, antibacterial and antifungal agents, isotonic and absorption delaying agents. A pharmaceutically acceptable excipient or carrier thus refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA (US Food and Drug Administration) or EMA (European Medicines Agency).
“Progression” in “multiple sclerosis progression (MS progression)” refers to the gradual worsening of symptoms and/or the gradual accumulation of disability occurring over time during the disease course. MS progression may for example be assessed with the Expanded Disability Status Scale (EDSS).
“Expanded Disability Status Scale (EDSS)” refers to a scale aiming at measuring the disability level of MS patients and at monitoring the changes in the disability level over time. It is commonly used for assessing the progression of the disease in patients with MS. The EDSS ranges from 0 to 10 with 0.5-unit increments, the lowest score 0 corresponding to a normal neurological exam without any disability and the highest score 10 corresponding to death due to MS.
“Progression of EDSS score” as used herein refers to the change over time in the EDSS score of a patient suffering from MS. As the disease progresses, the EDSS score increases. The progression of EDSS score may be used to assess the efficacy of a treatment. In one embodiment, the progression of EDSS score thus corresponds to the difference between the EDSS score at baseline (i.e., before treatment initiation) and the EDSS score once treatment has been initiated. The absence of difference in the EDSS score over time indicates an absence of disease progression and a stable state of the MS patient. A small increase (or a positive difference) in the EDSS score indicates a limited progression of the disease. Thus, the smaller the increase (i.e., the smaller the positive difference), the more stable the MS patient. Accordingly, a smaller increase in the EDSS score, as compared to the progression of EDSS score over an identical time period before treatment, indicates a slowing down of the progression of the disease. A decrease in the EDSS score (or a negative difference) indicates an absence of disease progression and an improvement of the patient. Thus, the greater the decrease (i.e., the greater the negative difference), the greater the improvement.
“Progressive multiple sclerosis (progressive MS)” as used herein refers to progressive forms of multiple sclerosis, that is to say forms of multiple sclerosis wherein symptoms gradually worsen and neurological function gradually deteriorate. Progressive forms of multiple sclerosis are thus characterized by a gradual decline over time, sometimes with extended periods without new or worsening symptoms, but without remission (i.e., no recovery or improvement once symptoms appear). Currently, multiple sclerosis (MS) is considered to encompass four types (also referred to as disease courses or phenotypes) as defined by the International Advisory Committee on Clinical Trials of MS in 2013. The four types of MS are:
   clinically isolated syndrome (CIS), defined as a first episode of neurologic symptoms caused by inflammation and demyelination in the central nervous system, which may or may not be followed by the development of MS;
   relapsing-remitting multiple sclerosis (RRMS or RR-MS), characterized by clearly defined relapses (also referred to as attacks, exacerbations or flare-ups) corresponding to episodes of new or worsening symptoms, followed by remissions corresponding to periods of partial or complete recovery or improvement with limited or no symptoms;
   secondary progressive multiple sclerosis (SPMS), which follows an initial relapsing-remitting phenotype and is characterized by a progressive worsening of symptoms and/or accumulation of disability over time, with (active SPMS) or without (non-active or relapse-free SPMS) the occurrence of relapses and remissions; and
   primary progressive multiple sclerosis (PPMS), characterized by a gradual worsening of symptoms and/or accumulation of disability from the onset of symptoms, without relapses or remissions.

In one particular embodiment, progressive multiple sclerosis (progressive MS) encompasses primary progressive multiple sclerosis (PPMS) and non-active secondary progressive multiple sclerosis (non-active SPMS).
“Primary progressive multiple sclerosis (PPMS)” refers to one of the four currently recognized types or phenotypes of multiple sclerosis. PPMS is characterized by a gradual worsening of symptoms and/or an accumulation of disability from the onset, without relapses (i.e., episodes of new or worsening symptoms) or remissions (i.e., periods of improvement or recovery with limited or no symptoms). According to one embodiment, PPMS is encompassed in progressive multiple sclerosis (progressive MS).
“Secondary progressive multiple sclerosis (SPMS)” refers to one of the four currently recognized types or phenotypes of multiple sclerosis. SPMS follows an initial course of relapsing-remitting multiple sclerosis (RRMS) and is characterized by a worsening of symptoms and/or an accumulation of disability over time.
“Non-active secondary progressive multiple sclerosis (non-active SPMS or nSPMS)” or “relapse-free secondary progressive multiple sclerosis (relapse-free SPMS)” refers to a subtype of SPMS. Non-active SPMS is characterized by the absence of relapses, i.e., of episodes of new or worsening symptoms. According to one embodiment, non-active (or relapse-free) SPMS is encompassed in progressive multiple sclerosis (progressive MS).
“Second-line” as in second-line treatment or second-line therapy for a disease or condition refers to a treatment or therapy administered after an initial treatment for said disease or condition (first-line treatment), usually because the initial treatment is ineffective or insufficiently effective, has stopped being effective, or has side effects. By contrast, “first-line” as in first-line treatment or first-line therapy for a disease or condition refers to the first or initial treatment or therapy administered for said disease or condition.
“Therapeutically effective amount” or “therapeutically effective dose” refers to the amount or dose or concentration of a 2-aminoarylthiazole derivative as described herein, in particular masitinib, or a pharmaceutically acceptable salt or solvate thereof, that is aimed at, without causing significant negative or adverse side-effects to the patient in need of treatment, preventing, reducing or slowing down (lessening) one or more of the symptoms or manifestations of progressive multiple sclerosis in said patient. In one embodiment, the “therapeutically effective amount” or “therapeutically effective dose” refers to the amount or dose or concentration of a 2-aminoarylthiazole derivative as described herein, in particular masitinib, or a pharmaceutically acceptable salt or solvate thereof, that is aimed at slowing down and/or limiting disease progression in a patient suffering from progressive MS, in particular at slowing down and/or limiting the worsening of symptoms and/or the gradual accumulation of disability. In one embodiment, the “therapeutically effective amount” or “therapeutically effective dose” refers to the amount or dose or concentration of a 2-aminoarylthiazole derivative as described herein, in particular masitinib, or a pharmaceutically acceptable salt or solvate thereof, that is aimed at bringing about a decrease of the EDSS score of a patient suffering from progressive MS or at bringing about a slowing down in the rate of increase of the EDSS score of a patient suffering from progressive MS.
“Treating” or “Treatment” refers to a therapeutic treatment, to a prophylactic (or preventative) treatment, or to both a therapeutic treatment and a prophylactic (or preventative) treatment, wherein the object is to prevent, reduce or slow down (lessen) one or more of the symptoms or manifestations of progressive MS in a patient in need thereof, preferably the object is to slow down and/or limit disease progression in a patient suffering from progressive MS, and in particular to slow down and/or limit the worsening of symptoms and/or the gradual accumulation of disability in a patient suffering from progressive MS. In one embodiment, the object of the treatment is to bring about a decrease of the EDSS score of a patient suffering from progressive MS or to bring about a slowing down in the rate of increase of the EDSS score of a patient suffering from progressive MS.
“Time from diagnosis to treatment initiation” as used herein refers to the length of time between date of first clinically definite diagnosis for MS, in particular for progressive MS (expressed as day/month/year, month/year, or year) and date of treatment initiation with the 2-aminoarylthiazole derivative as described herein, in particular masitinib, or a pharmaceutically acceptable salt or solvate thereof (expressed as day/month/year, month/year, or year). In one embodiment, diagnosis of progressive MS is based on the revised McDonald criteria. At the time of diagnosis of MS, a provisional disease course should be specified and whether the course is active or not (non-active) and progressive or not based on the previous year's history. In one embodiment, if the date is expressed as month/year (i.e., if information regarding the day is missing), the day is considered to be the last day of the month. In one embodiment, if the date is expressed as a year (i.e., if information regarding the day and the month are missing), the month is considered to be June and the day is considered to be the last day of the month, i.e., the last day of June.
“Time from onset to treatment initiation” as used herein refers to the length of time between date of first MS-related symptom(s) (expressed as day/month/year, month/year, or year) and date of treatment initiation with the 2-aminoarylthiazole derivative as described herein, in particular masitinib, or a pharmaceutically acceptable salt or solvate thereof (expressed as day/month/year, month/year, or year). In one embodiment, the date of first symptom(s) is determined retrospectively from medical history, for example from medical records and patient/caregiver historical recall of MS-related symptom(s) following clinically definite diagnosis. In one embodiment, if the date is expressed as month/year (i.e., if information regarding the day is missing), the day is considered to be the last day of the month. In one embodiment, if the date is expressed as a year (i.e., if information regarding the day and the month are missing), the month is considered to be June and the day is considered to be the last day of the month, i.e., the last day of June.

### DETAILED DESCRIPTION

The present invention relates to a 2-aminoarylthiazole derivative as described herein, in particular masitinib, or a pharmaceutically acceptable salt or solvate thereof, for use in the treatment of progressive multiple sclerosis (MS) in a patient in need thereof.

According to the present invention, progressive MS refers to progressive forms of MS, characterized by a gradual decline over time, sometimes with extended periods without new or worsening symptoms, and by the absence of remission (i.e., no recovery or improvement once symptoms appear).

In one embodiment, progressive MS comprises primary progressive multiple sclerosis (PPMS) and non-active secondary progressive multiple sclerosis (non-active SPMS). In one embodiment, progressive MS consists of PPMS and non-active SPMS.

In one embodiment, progressive MS is diagnosed using the 2017 revised McDonald criteria. The 2017 revised McDonald criteria for the diagnosis of MS is a well-established diagnostic instrument that is widely used in research and clinical practice. The McDonald criteria for the diagnosis of multiple sclerosis have been published in Polman et al., Ann Neurol. 2011; 69(2):292-302. The 2017 revisions of the McDonald criteria have been published in Thompson et al., Lancet Neurol. 2018; 17(2):162-173. According to one embodiment, a patient who was diagnosed as suffering from MS using previous versions of the McDonald criteria will also meet the criteria for MS as laid out in the 2017 McDonald criteria.

Briefly, the 2010 McDonald criteria for diagnosis of MS as published in Polman et al., Ann Neurol. 2011; 69(2):292-302 are shown below in Table 1.

TABLE 1
2010 McDonald criteria for diagnosis of MS

| Clinical Presentation | Additional data need for MS diagnosis |
|---|---|
| ≥2 attacks^{a}; objective clinical evidence | None^{c} |
| of ≥2 lesions or objective clinical evidence | |
| of 1 lesion with reasonable historical | |
| evidence of a prior attack^{b} | |
| ≥2 attacks^{a}; objective clinical evidence of | Dissemination in space, demonstrated by: ≥1 |
| 1 lesion | T2 lesion in at least 2 of 4 MS-typical |
| | regions of the CNS (periventricular, |
| | juxtacortical, infratentorial, or spinal cord)^{d}; or |
| | Await a further clinical attack^{a} implicating |
| | a different CNS site |
| 1 attack^{a}; objective clinical evidence | Dissemination in time, demonstrated by: |
| of ≥2 lesions | Simultaneous presence of asymptomatic |
| | gadolinium-enhancing and nonenhancing |
| | lesions at any time; or |
| | A new T2 and/or gadolinium-enhancing |
| | lesion(s) on follow-up MRI, irrespective |
| | of its timing with reference to a baseline scan; or |
| | Await a second clinical attack^{a} |
| 1 attack^{a}; objective clinical evidence of 1 | Dissemination in space and time, demonstrated |
| lesion (clinically isolated syndrome) | by: For DIS: ≥1 T2 |
| | lesion in at least 2 of 4 MS-typical |
| | regions of the CNS (periventricular, |
| | juxtacortical, infratentorial, or spinal cord)^{d}; or |
| | Await a second clinical attack^{a} implicating |
| | a different CNS site; and For DIT: |
| | Simultaneous presence of asymptomatic |
| | gadolinium-enhancing and nonenhancing |
| | lesions at any time; or |
| | A new T2 and/or gadolinium-enhancing |
| | lesion(s) on follow-up MRI, irrespective |
| | of its timing with reference to a baseline scan; or |
| | Await a second clinical attack^{a} |
| Insidious neurological progression | 1 year of disease progression |
| suggestive of MS (PPMS) | (retrospectively or prospectively |
| | determined) plus 2 of 3 of the following criteria^{d}: |
| | 1. Evidence for DIS in the brain based |
| | on ≥1 T2 lesions in the MS-characteristic |
| | (periventricular, juxtacortical,or |
| | infratentorial) regions; |
| | 2. Evidence for DIS in the spinal cord |
| | based on ≥2 T2 lesions in the cord; |
| | 3. Positive CSF (isoelectric focusing |
| | evidence of oligoclonal bands and/or |
| | elevated IgG index) |

^{a}An attack (also known as relapse, or exacerbation) is defined as patient-reported or objectively observed event(s) typical of an acute inflammatory demyelinating event in the CNS, current or historical, with duration of at least 24 hours, in the absence of fever or infection.
^{b}Clinical diagnosis based on objective clinical findings for 2 attacks is most secure.
^{c}No additional tests are required.
^{d}Gadolinium-enhancing lesions are not required; symptomatic lesions are excluded from consideration in subjects with brainstem or spinal cord syndromes.
MS: multiple sclerosis;
CNS: central nervous system;
MRI: magnetic resonance imaging;
DIS: dissemination in space;
DIT: dissemination in time;
PPMS: primary progressive multiple sclerosis;
CSF: cerebrospinal fluid;
IgG: immunoglobulin G.

The revised 2017 McDonald criteria for diagnosis of MS in patients with an attack at onset (i.e., relapsing-remitting multiple sclerosis or RRMS) as published in Thompson et al., Lancet Neurol. 2018; 17(2):162-173 are shown below in Table 2.

TABLE 2
2017 revised McDonald criteria for diagnosis of MS in patients with an
attack at onset

| | Number of lesions with | Additional data needed for a |
|---|---|---|
| | objective clinical evidence | diagnosis of multiple sclerosis |
| ≥2 clinical | ≥2 | None* |
| attacks | | |
| ≥2 clinical | 1 (as well as clear-cut historical | None* |
| attacks | evidence of a previous attack | |
| | involving a lesion in a | |
| | distinct anatomical location^{†}) | |
| ≥2 clinical | 1 | Dissemination in space |
| attacks | | demonstrated by an additional |
| | | clinical attack implicating a |
| | | different CNS site or by MRI^{‡} |
| 1 clinical attack | ≥2 | Dissemination in time demonstrated |
| | | by an additional clinical attack or by |
| | | MRI^{§} OR demonstration of |
| | | CSF-specific oligoclonal bands^{¶} |
| 1 clinical attack | 1 | Dissemination in space |
| | | demonstrated by an additional |
| | | clinical attack implicating a |
| | | different CNS site or by MRI^{‡} AND |
| | | Dissemination in time demonstrated |
| | | by an additional clinical attack or by |
| | | MRI^{§} OR demonstration of |
| | | CSF-specific oligoclonal bands^{‡} |

*No additional tests are required to demonstrate dissemination in space and time.
^{†}Clinical diagnosis based on objective clinical findings for two attacks is most secure.
^{‡}The MRI criteria for dissemination in space are: one or more T2-hyperintense lesions that are characteristic of multiple sclerosis in two or more of four areas of the CNS: periventricular, cortical or juxtacortical, and infratentorial brain regions, and the spinal cord.
^{§}Dissemination in space can be demonstrated by the simultaneous presence of gadolinium-enhancing and non-enhancing lesions at any time or by a new T2-hyperintense or gadolinium-enhancing lesion on follow-up MRI, with reference to a baseline scan, irrespective of the timing of the baseline MRI.
^{¶}The presence of CSF-specific oligoclonal bands does not demonstrate dissemination in time per se but can substitute for the requirement for demonstration of this measure.

The revised 2017 McDonald criteria for diagnosis of MS in patients with disease course characterized by progression from onset (i.e., primary progressive multiple sclerosis or PPMS) as published in Thompson et al., Lancet Neurol. 2018; 17(2):162-173 are as follows:
1 year of disability progression (retrospectively or prospectively determined) independent of clinical relapse; and
Two of the following criteria:
   One or more T2-hyperintense lesion(s) characteristic of multiple sclerosis in one or more of the following brain region(s): periventricular, cortical or juxtacortical, or infratentorial;
   Two or more T2-hyperintense lesions in the spinal cord;
   Presence of CSF-specific oligoclonal bands.

According to one embodiment, the present invention relates to a 2-aminoarylthiazole derivative as described herein, or a pharmaceutically acceptable salt or solvate thereof, for use in the treatment of progressive MS in a patient in need thereof, wherein said patient has a time from diagnosis to treatment initiation with the 2-aminoarylthiazole derivative, or a pharmaceutically acceptable salt or solvate thereof, greater than 1 year and/or has a time from onset to treatment initiation with the 2-aminoarylthiazole derivative, or a pharmaceutically acceptable salt or solvate thereof, greater than 2 years.

Accordingly, in one embodiment, the present invention relates to a 2-aminoarylthiazole derivative as described herein, or a pharmaceutically acceptable salt or solvate thereof, for use in the treatment of progressive MS in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 1 year or more from diagnosis (i.e., from the time of diagnosis) and/or 2 years or more from disease onset (i.e., from the time of disease onset).

In one embodiment, the present invention relates to a 2-aminoarylthiazole derivative as described herein, or a pharmaceutically acceptable salt or solvate thereof, for use in the treatment of progressive MS in a patient in need thereof, wherein said patient has a time from diagnosis to treatment initiation with the 2-aminoarylthiazole derivative, or a pharmaceutically acceptable salt or solvate thereof, greater than 2 years and/or has a time from onset to treatment initiation with the 2-aminoarylthiazole derivative, or a pharmaceutically acceptable salt or solvate thereof, greater than 5 years. In one embodiment, the present invention relates to a 2-aminoarylthiazole derivative as described herein, or a pharmaceutically acceptable salt or solvate thereof, for use in the treatment of progressive MS in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 2 years or more from diagnosis (i.e., from the time of diagnosis) and/or 5 years or more from disease onset (i.e., from the time of disease onset).

In one embodiment, the present invention relates to a 2-aminoarylthiazole derivative as described herein, or a pharmaceutically acceptable salt or solvate thereof, for use in the treatment of progressive MS in a patient in need thereof, wherein said patient has a time from diagnosis to treatment initiation with the 2-aminoarylthiazole derivative, or a pharmaceutically acceptable salt or solvate thereof, greater than 3 years and/or has a time from onset to treatment initiation with the 2-aminoarylthiazole derivative, or a pharmaceutically acceptable salt or solvate thereof, greater than 5 years. In one embodiment, the present invention relates to a 2-aminoarylthiazole derivative as described herein, or a pharmaceutically acceptable salt or solvate thereof, for use in the treatment of progressive MS in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 3 years or more from diagnosis (i.e., from the time of diagnosis) and/or 5 years or more from disease onset (i.e., from the time of disease onset).

According to the present invention, the time from diagnosis to treatment initiation with the 2-aminoarylthiazole derivative as described herein, or a pharmaceutically acceptable salt or solvate thereof, is the length of time between date of first clinically definite diagnosis for MS, in particular for progressive MS (expressed as day/month/year, month/year, or year) and date of treatment initiation with said 2-aminoarylthiazole derivative, or a pharmaceutically acceptable salt or solvate thereof (expressed as day/month/year, month/year, or year). Accordingly, the time of diagnosis is the date of first clinically definite diagnosis for MS, in particular for progressive MS (expressed as day/month/year, month/year, or year).

In one embodiment, the time from diagnosis to treatment initiation with the 2-aminoarylthiazole derivative as described herein, or a pharmaceutically acceptable salt or solvate thereof, is the length of time between date of first clinically definite diagnosis for progressive MS and date of treatment initiation with said 2-aminoarylthiazole derivative, or a pharmaceutically acceptable salt or solvate thereof. Accordingly, in one embodiment, the time of diagnosis is the date of first clinically definite diagnosis for progressive MS.

In one embodiment, the time from diagnosis to treatment initiation with the 2-aminoarylthiazole derivative as described herein, or a pharmaceutically acceptable salt or solvate thereof, is the length of time between date of first clinically definite diagnosis for progressive MS based on the 2017 revised McDonald criteria and date of treatment initiation with said 2-aminoarylthiazole derivative, or a pharmaceutically acceptable salt or solvate thereof. Accordingly, in one embodiment, the time of diagnosis is the date of first clinically definite diagnosis for progressive MS based on the 2017 revised McDonald criteria.

In one embodiment, if the date is expressed as month/year (i.e., if information regarding the day is missing), the day is considered to be the last day of the month. In one embodiment, if the date is expressed as a year (i.e., if information regarding the day and the month are missing), the month is considered to be June and the day is considered to be the last day of the month, i.e., the last day of June.

In one embodiment, the patient has a time from diagnosis to treatment initiation with the 2-aminoarylthiazole derivative, or a pharmaceutically acceptable salt or solvate thereof, greater than 1 year, greater than 2 years, greater than 3 years, greater than 4 years, greater than 5 years, or greater than 6 years. In one embodiment, the patient has a time from diagnosis to treatment initiation with the 2-aminoarylthiazole derivative, or a pharmaceutically acceptable salt or solvate thereof, greater than 2 years. In one embodiment, the patient has a time from diagnosis to treatment initiation with the 2-aminoarylthiazole derivative, or a pharmaceutically acceptable salt or solvate thereof, greater than 3 years.

In one embodiment, the 2-aminoarylthiazole derivative, or a pharmaceutically acceptable salt or solvate thereof, is for administration, optionally as a further or second-line treatment and/or as an add-on to supportive care or to palliative care, 1 year or more, 2 years or more, 3 years or more, 4 years or more, 5 years or more, or 6 years or more from diagnosis (i.e., from the time of diagnosis). In one embodiment, the 2-aminoarylthiazole derivative, or a pharmaceutically acceptable salt or solvate thereof, is for administration, optionally as a further or second-line treatment and/or as an add-on to supportive care or to palliative care, 2 years or more from diagnosis (i.e., from the time of diagnosis). In one embodiment, the 2-aminoarylthiazole derivative, or a pharmaceutically acceptable salt or solvate thereof, is for administration, optionally as a further or second-line treatment and/or as an add-on to supportive care or to palliative care, 3 years or more from diagnosis (i.e., from the time of diagnosis).

According to the present invention, the time from onset to treatment initiation with the 2-aminoarylthiazole derivative as described herein, or a pharmaceutically acceptable salt or solvate thereof, is the length of time between date of first MS-related symptom(s) (expressed as day/month/year, month/year, or year) and date of treatment initiation with said 2-aminoarylthiazole derivative, or a pharmaceutically acceptable salt or solvate thereof (expressed as day/month/year, month/year, or year). Accordingly, the time of disease onset is the date of first MS-related symptom(s) (expressed as day/month/year, month/year, or year).

In one embodiment, the date of first symptom(s) is determined retrospectively from medical history, for example from medical records and patient/caregiver historical recall of MS-related symptom(s) following clinically definite diagnosis. In one embodiment, if the date is expressed as month/year (i.e., if information regarding the day is missing), the day is considered to be the last day of the month. In one embodiment, if the date is expressed as a year (i.e., if information regarding the day and the month are missing), the month is considered to be June and the day is considered to be the last day of the month, i.e., the last day of June.

In one embodiment, first MS-related symptom(s) is/are first progressive MS-related symptom(s). In one embodiment, the date of first progressive MS-related symptom(s) is the timepoint associated with irreversible disability accumulation or irreversible progression of the disease. In one embodiment, irreversible disability accumulation or irreversible progression of the disease is defined by an EDSS score that does not revert to zero at a later follow-up. In one embodiment, irreversible disability accumulation or irreversible progression of the disease is defined as a sustained elevated EDSS score of at least 0.5, at least 1.0, or at least 1.5, that does not revert to zero at a later follow-up.

In one embodiment, the patient has a time from onset to treatment initiation with the 2-aminoarylthiazole derivative, or a pharmaceutically acceptable salt or solvate thereof, greater than 2 years, greater than 3 years, greater than 4 years, greater than 5 years, greater than 6 years, or greater than 7 years. In one embodiment, the patient has a time from onset to treatment initiation with the 2-aminoarylthiazole derivative, or a pharmaceutically acceptable salt or solvate thereof, greater than 5 years.

In one embodiment, the 2-aminoarylthiazole derivative, or a pharmaceutically acceptable salt or solvate thereof, is for administration, optionally as a further or second-line treatment and/or as an add-on to supportive care or to palliative care, 2 years or more, 3 years or more, 4 years or more, 5 years or more, 6 years or more, or 7 years or more from disease onset (i.e., from the time of disease onset). In one embodiment, the 2-aminoarylthiazole derivative, or a pharmaceutically acceptable salt or solvate thereof, is for administration, optionally as a further or second-line treatment and/or as an add-on to supportive care or to palliative care, 5 years or more from disease onset (i.e., from the time of disease onset).

In one embodiment, the patient has a time from diagnosis to treatment initiation with the 2-aminoarylthiazole derivative, or a pharmaceutically acceptable salt or solvate thereof, greater than 1 year, greater than 2 years, greater than 3 years, greater than 4 years, greater than 5 years, or greater than 6 years and said patient has a time from onset to treatment initiation with the 2-aminoarylthiazole derivative, or a pharmaceutically acceptable salt or solvate thereof, greater than 2 years, greater than 3 years, greater than 4 years, greater than 5 years, greater than 6 years, or greater than 7 years.

In one embodiment, the 2-aminoarylthiazole derivative, or a pharmaceutically acceptable salt or solvate thereof, is for administration, optionally as a further or second-line treatment and/or as an add-on to supportive care or to palliative care, 1 year or more, 2 years or more, 3 years or more, 4 years or more, 5 years or more, or 6 years or more from diagnosis (i.e., from the time of diagnosis) and 2 years or more, 3 years or more, 4 years or more, 5 years or more, 6 years or more, or 7 years or more from disease onset (i.e., from the time of disease onset).

In one embodiment, the patient has a time from diagnosis to treatment initiation with the 2-aminoarylthiazole derivative, or a pharmaceutically acceptable salt or solvate thereof, greater than 1 year and has a time from onset to treatment initiation with the 2-aminoarylthiazole derivative, or a pharmaceutically acceptable salt or solvate thereof, greater than 5 years. In one embodiment, the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 4 years. In one embodiment, the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 5 years. In one embodiment, the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 6 years. In one embodiment, the patient has a time from diagnosis to treatment initiation with the 2-aminoarylthiazole derivative, or a pharmaceutically acceptable salt or solvate thereof, greater than 3 years and has a time from onset to treatment initiation with the 2-aminoarylthiazole derivative, or a pharmaceutically acceptable salt or solvate thereof, greater than 5 years.

In one embodiment, the 2-aminoarylthiazole derivative, or a pharmaceutically acceptable salt or solvate thereof, is for administration, optionally as a further or second-line treatment and/or as an add-on to supportive care or to palliative care, 1 year or more from diagnosis (i.e., from the time of diagnosis) and 5 years or more from disease onset (i.e., from the time of disease onset). In one embodiment, the 2-aminoarylthiazole derivative, or a pharmaceutically acceptable salt or solvate thereof, is for administration, optionally as a further or second-line treatment and/or as an add-on to supportive care or to palliative care, 2 years or more from diagnosis (i.e., from the time of diagnosis) and 4 years or more from disease onset (i.e., from the time of disease onset) In one embodiment, the 2-aminoarylthiazole derivative, or a pharmaceutically acceptable salt or solvate thereof, is for administration, optionally as a further or second-line treatment and/or as an add-on to supportive care or to palliative care, 2 years or more from diagnosis (i.e., from the time of diagnosis) and 5 years or more from disease onset (i.e., from the time of disease onset). In one embodiment, the 2-aminoarylthiazole derivative, or a pharmaceutically acceptable salt or solvate thereof, is for administration, optionally as a further or second-line treatment and/or as an add-on to supportive care or to palliative care, 2 years or more from diagnosis (i.e., from the time of diagnosis) and 6 years or more from disease onset (i.e., from the time of disease onset). In one embodiment, the 2-aminoarylthiazole derivative, or a pharmaceutically acceptable salt or solvate thereof, is for administration, optionally as a further or second-line treatment and/or as an add-on to supportive care or to palliative care, 3 years or more from diagnosis (i.e., from the time of diagnosis) and 5 years or more from disease onset (i.e., from the time of disease onset).

According to one embodiment, progressive multiple sclerosis is primary progressive multiple sclerosis (PPMS).

Thus, in one embodiment, the present invention relates to a 2-aminoarylthiazole derivative as described herein, or a pharmaceutically acceptable salt or solvate thereof, for use in the treatment of PPMS in a patient in need thereof, wherein said patient has a time from diagnosis to treatment initiation with the 2-aminoarylthiazole derivative, or a pharmaceutically acceptable salt or solvate thereof, greater than 1 year and/or has a time from onset to treatment initiation with the 2-aminoarylthiazole derivative, or a pharmaceutically acceptable salt or solvate thereof, greater than 2 years. In one embodiment, the present invention relates to a 2-aminoarylthiazole derivative as described herein, or a pharmaceutically acceptable salt or solvate thereof, for use in the treatment of PPMS in a patient in need thereof, wherein said patient has a time from diagnosis to treatment initiation with the 2-aminoarylthiazole derivative, or a pharmaceutically acceptable salt or solvate thereof, greater than 2 years and/or has a time from onset to treatment initiation with the 2-aminoarylthiazole derivative, or a pharmaceutically acceptable salt or solvate thereof, greater than 5 years. In one embodiment, the present invention relates to a 2-aminoarylthiazole derivative as described herein, or a pharmaceutically acceptable salt or solvate thereof, for use in the treatment of PPMS in a patient in need thereof, wherein said patient has a time from diagnosis to treatment initiation with the 2-aminoarylthiazole derivative, or a pharmaceutically acceptable salt or solvate thereof, greater than 3 years and/or has a time from onset to treatment initiation with the 2-aminoarylthiazole derivative, or a pharmaceutically acceptable salt or solvate thereof, greater than 5 years.

In one embodiment, the present invention relates to a 2-aminoarylthiazole derivative as described herein, or a pharmaceutically acceptable salt or solvate thereof, for use in the treatment of PPMS in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 1 year or more from diagnosis (i.e., from the time of diagnosis) and/or 2 years or more from disease onset (i.e., from the time of disease onset). In one embodiment, the present invention relates to a 2-aminoarylthiazole derivative as described herein, or a pharmaceutically acceptable salt or solvate thereof, for use in the treatment of PPMS in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 2 years or more from diagnosis (i.e., from the time of diagnosis) and/or 5 years or more from disease onset (i.e., from the time of disease onset). In one embodiment, the present invention relates to a 2-aminoarylthiazole derivative as described herein, or a pharmaceutically acceptable salt or solvate thereof, for use in the treatment of PPMS in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 3 years or more from diagnosis (i.e., from the time of diagnosis) and/or 5 years or more from disease onset (i.e., from the time of disease onset).

Accordingly, in one embodiment, the time from diagnosis to treatment initiation with the 2-aminoarylthiazole derivative as described herein, or a pharmaceutically acceptable salt or solvate thereof, is the length of time between date of first clinically definite diagnosis for PPMS (expressed as day/month/year, month/year, or year) and date of treatment initiation with said 2-aminoarylthiazole derivative, or a pharmaceutically acceptable salt or solvate thereof (expressed as day/month/year, month/year, or year). Accordingly, in one embodiment, the time of diagnosis is the date of first clinically definite diagnosis for PPMS.

In one embodiment, the time from diagnosis to treatment initiation with the 2-aminoarylthiazole derivative as described herein, or a pharmaceutically acceptable salt or solvate thereof, is the length of time between date of first clinically definite diagnosis for PPMS based on the 2017 revised McDonald criteria and date of treatment initiation with said 2-aminoarylthiazole derivative, or a pharmaceutically acceptable salt or solvate thereof. Accordingly, in one embodiment, the time of diagnosis is the date of first clinically definite diagnosis for PPMS based on the 2017 revised McDonald criteria.

In one embodiment, progressive multiple sclerosis is PPMS, and the patient has a time from diagnosis to treatment initiation with the 2-aminoarylthiazole derivative, or a pharmaceutically acceptable salt or solvate thereof, greater than 1 year, greater than 2 years, greater than 3 years, greater than 4 years, greater than 5 years, or greater than 6 years. In one embodiment, progressive multiple sclerosis is PPMS, and the patient has a time from diagnosis to treatment initiation with the 2-aminoarylthiazole derivative, or a pharmaceutically acceptable salt or solvate thereof, greater than 2 years. In one embodiment, progressive multiple sclerosis is PPMS, and the patient has a time from diagnosis to treatment initiation with the 2-aminoarylthiazole derivative, or a pharmaceutically acceptable salt or solvate thereof, greater than 3 years.

In one embodiment, progressive multiple sclerosis is PPMS, and the patient has a time from onset to treatment initiation with the 2-aminoarylthiazole derivative, or a pharmaceutically acceptable salt or solvate thereof, greater than 2 years, greater than 3 years, greater than 4 years, greater than 5 years, greater than 6 years, or greater than 7 years. In one embodiment, progressive multiple sclerosis is PPMS, and the patient has a time from onset to treatment initiation with the 2-aminoarylthiazole derivative, or a pharmaceutically acceptable salt or solvate thereof, greater than 5 years.

In one embodiment, progressive multiple sclerosis is PPMS, and the patient has a time from diagnosis to treatment initiation with the 2-aminoarylthiazole derivative, or a pharmaceutically acceptable salt or solvate thereof, greater than 1 year, greater than 2 years, greater than 3 years, greater than 4 years, greater than 5 years, or greater than 6 years and said patient has a time from onset to treatment initiation with the 2-aminoarylthiazole derivative, or a pharmaceutically acceptable salt or solvate thereof, greater than 2 years, greater than 3 years, greater than 4 years, greater than 5 years, greater than 6 years, or greater than 7 years.

In one embodiment, progressive multiple sclerosis is PPMS, and the patient has a time from diagnosis to treatment initiation with the 2-aminoarylthiazole derivative, or a pharmaceutically acceptable salt or solvate thereof, greater than 1 year and has a time from onset to treatment initiation with the 2-aminoarylthiazole derivative, or a pharmaceutically acceptable salt or solvate thereof, greater than 5 years. In one embodiment, progressive multiple sclerosis is PPMS, and the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 4 years. In one embodiment, progressive multiple sclerosis is PPMS, and the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 5 years. In one embodiment, progressive multiple sclerosis is PPMS, and the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 6 years. In one embodiment, progressive multiple sclerosis is PPMS, and the patient has a time from diagnosis to treatment initiation with the 2-aminoarylthiazole derivative, or a pharmaceutically acceptable salt or solvate thereof, greater than 3 years and has a time from onset to treatment initiation with the 2-aminoarylthiazole derivative, or a pharmaceutically acceptable salt or solvate thereof, greater than 5 years.

According to one embodiment, progressive multiple sclerosis is non-active secondary progressive multiple sclerosis (non-active SPMS or nSPMS).

Thus, in one embodiment, the present invention relates to a 2-aminoarylthiazole derivative as described herein, or a pharmaceutically acceptable salt or solvate thereof, for use in the treatment of non-active SPMS (nSPMS) in a patient in need thereof, wherein said patient has a time from diagnosis to treatment initiation with the 2-aminoarylthiazole derivative, or a pharmaceutically acceptable salt or solvate thereof, greater than 1 year and/or has a time from onset to treatment initiation with the 2-aminoarylthiazole derivative, or a pharmaceutically acceptable salt or solvate thereof, greater than 2 years. In one embodiment, the present invention relates to a 2-aminoarylthiazole derivative as described herein, or a pharmaceutically acceptable salt or solvate thereof, for use in the treatment of non-active SPMS (nSPMS) in a patient in need thereof, wherein said patient has a time from diagnosis to treatment initiation with the 2-aminoarylthiazole derivative, or a pharmaceutically acceptable salt or solvate thereof, greater than 2 years and/or has a time from onset to treatment initiation with the 2-aminoarylthiazole derivative, or a pharmaceutically acceptable salt or solvate thereof, greater than 5 years. In one embodiment, the present invention relates to a 2-aminoarylthiazole derivative as described herein, or a pharmaceutically acceptable salt or solvate thereof, for use in the treatment of non-active SPMS (nSPMS) in a patient in need thereof, wherein said patient has a time from diagnosis to treatment initiation with the 2-aminoarylthiazole derivative, or a pharmaceutically acceptable salt or solvate thereof, greater than 3 years and/or has a time from onset to treatment initiation with the 2-aminoarylthiazole derivative, or a pharmaceutically acceptable salt or solvate thereof, greater than 5 years.

In one embodiment, the present invention relates to a 2-aminoarylthiazole derivative as described herein, or a pharmaceutically acceptable salt or solvate thereof, for use in the treatment of non-active SPMS (nSPMS) in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 1 year or more from diagnosis (i.e., from the time of diagnosis) and/or 2 years or more from disease onset (i.e., from the time of disease onset). In one embodiment, the present invention relates to a 2-aminoarylthiazole derivative as described herein, or a pharmaceutically acceptable salt or solvate thereof, for use in the treatment of non-active SPMS (nSPMS) in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 2 years or more from diagnosis (i.e., from the time of diagnosis) and/or 5 years or more from disease onset (i.e., from the time of disease onset). In one embodiment, the present invention relates to a 2-aminoarylthiazole derivative as described herein, or a pharmaceutically acceptable salt or solvate thereof, for use in the treatment of non-active SPMS (nSPMS) in a patient in need thereof, wherein said 2-aminoarylthiazole derivative, or a pharmaceutically acceptable salt or solvate thereof, is for administration, optionally as a further or second-line treatment and/or as an add-on to supportive care or to palliative care, 3 years or more from diagnosis (i.e., from the time of diagnosis) and/or 5 years or more from disease onset (i.e., from the time of disease onset).

Accordingly, in one embodiment, the time from diagnosis to treatment initiation with the 2-aminoarylthiazole derivative as described herein, or a pharmaceutically acceptable salt or solvate thereof, is the length of time between date of first clinically definite diagnosis for non-active SPMS or nSPMS (expressed as day/month/year, month/year, or year) and date of treatment initiation with said 2-aminoarylthiazole derivative, or a pharmaceutically acceptable salt or solvate thereof (expressed as day/month/year, month/year, or year). Accordingly, in one embodiment, the time of diagnosis is the date of first clinically definite diagnosis for non-active SPMS or nSPMS.

In one embodiment, the time from diagnosis to treatment initiation with the 2-aminoarylthiazole derivative as described herein, or a pharmaceutically acceptable salt or solvate thereof, is the length of time between date of first clinically definite diagnosis for non-active SPMS or nSPMS based on the 2017 revised McDonald criteria and date of treatment initiation with said 2-aminoarylthiazole derivative, or a pharmaceutically acceptable salt or solvate thereof. Accordingly, in one embodiment, the time of diagnosis is the date of first clinically definite diagnosis for non-active SPMS or nSPMS based on the 2017 revised McDonald criteria.

In one embodiment, progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 1 year, greater than 2 years, greater than 3 years, greater than 4 years, greater than 5 years, or greater than 6 years. In one embodiment, progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 2 years. In one embodiment, progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 3 years.

In one embodiment, progressive multiple sclerosis is non-active SPMS (nSPMS), and the patient has a time from onset to treatment initiation with the 2-aminoarylthiazole derivative, or a pharmaceutically acceptable salt or solvate thereof, greater than 2 years, greater than 3 years, greater than 4 years, greater than 5 years, greater than 6 years, or greater than 7 years. In one embodiment, progressive multiple sclerosis is non-active SPMS (nSPMS), and the patient has a time from onset to treatment initiation with the 2-aminoarylthiazole derivative, or a pharmaceutically acceptable salt or solvate thereof, greater than 5 years.

In one embodiment, progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 1 year, greater than 2 years, greater than 3 years, greater than 4 years, greater than 5 years, or greater than 6 years and said patient has a time from onset to treatment initiation with the 2-aminoarylthiazole derivative, or a pharmaceutically acceptable salt or solvate thereof, greater than 2 years, greater than 3 years, greater than 4 years, greater than 5 years, greater than 6 years, or greater than 7 years.

In one embodiment, progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 1 year and has a time from onset to treatment initiation with the 2-aminoarylthiazole derivative, or a pharmaceutically acceptable salt or solvate thereof, greater than 5 years. In one embodiment progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 4 years In one embodiment, progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 5 years. In one embodiment, progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 2 years and has a time from onset to treatment initiation with the 2-aminoarylthiazole derivative, or a pharmaceutically acceptable salt or solvate thereof, greater than 6 years. In one embodiment, progressive multiple sclerosis is non-active SPMS (nSPMS), and the patient has a time from diagnosis to treatment initiation with the 2-aminoarylthiazole derivative, or a pharmaceutically acceptable salt or solvate thereof, greater than 3 years and has a time from onset to treatment initiation with the 2-aminoarylthiazole derivative, or a pharmaceutically acceptable salt or solvate thereof, greater than 5 years.

According to the present invention, “treatment” refers to a therapeutic treatment, to a prophylactic (or preventative) treatment, or to both a therapeutic treatment and a prophylactic (or preventative) treatment, wherein the object is to prevent, reduce or slow down (lessen) one or more of the symptoms or manifestations of progressive multiple sclerosis in a patient in need thereof.

In one embodiment, the object of the treatment is to slow down and/or limit disease progression in a patient suffering from progressive MS. In one embodiment, the object of the treatment is thus to slow down and/or limit the worsening of symptoms and/or the gradual accumulation of disability in a patient suffering from progressive MS.

According to one embodiment, disease progression in the patient suffering from progressive MS is assessed with the Expanded Disability Status Scale (EDSS).

As mentioned hereinabove, the EDSS is a scale aiming at measuring the disability level of MS patients and at monitoring the changes in the disability level over time (Kurtzke J F. Neurology. 1983; 33(11):1444-1452). The EDSS is based on a neurological examination by a physician, and ranges from 0 to 10 with 0.5-unit increments, the lowest score 0 corresponding to a normal neurological exam without any disability and the highest score 10 corresponding to death due to MS (see Table 3 below).

Patients with EDSS scores ranging from 1.0 to 4.5 are able to walk without any aid. The score assessment is based on measures of impairment in 8 functional systems (FS), each representing a network of neurons with responsibility for particular tasks and each being scored on a scale of 0 (no disability) to 5 or 6 (more severe disability). The 8 FS are: pyramidal (muscle weakness or difficulty moving limbs); cerebellar (ataxia, loss of balance, coordination or tremor); brainstem (problems with speech, swallowing and nystagmus); sensory (numbness or loss of sensations); bowel and bladder function;

visual function; cerebral functions; and other.

Patients with EDSS scores ranging from 5 to 9.5 are identified by the impairment to walking.

TABLE 3
Expanded Disability Status Scale (EDSS)

| Score | Description |
|---|---|
| 0 | Normal neurological exam, no disability in any FS |
| 1.0 | No disability, minimal signs in one FS |
| 1.5 | No disability, minimal signs in more than one FS |
| 2.0 | Minimal disability in one FS |
| 2.5 | Mild disability in one FS or minimal disability in two FS |
| 3.0 | Moderate disability in one FS, or mild disability in three or four FS. No impairment to walking |
| 3.5 | Moderate disability in one FS and more than minimal disability in several others. No impairment to walking |
| 4.0 | Significant disability but self-sufficient and up and about some 12 hours |
| | a day. Able to walk without aid or rest for 500 m |
| 4.5 | Significant disability but up and about much of the day, able to work a full day, may otherwise have some |
| | limitation of full activity or require minimal assistance. Able to walk without aid or rest for 300 m |
| 5.0 | Disability severe enough to impair full daily activities and ability to work |
| | a full day without special provisions. Able to walk without aid or rest for 200 m |
| 5.5 | Disability severe enough to preclude full daily activities. Able to walk without aid or rest for 100 m |
| 6.0 | Requires a walking aid - cane, crutch, etc. - to walk about 100 m with or without resting |
| 6.5 | Requires two walking aids - pair of canes, crutches, etc. - to walk about 20 m without resting |
| 7.0 | Unable to walk beyond approximately 5 m even with aid. Essentially restricted to wheelchair; |
| | though wheels self in standard wheelchair and transfers alone. Up and about in wheelchair some 12 hours a day |
| 7.5 | Unable to take more than a few steps. Restricted to wheelchair and may need aid in transferring. |
| | Can wheel self but cannot carry on in standard wheelchair for a full day and may require a motorized wheelchair |
| 8.0 | Essentially restricted to bed or chair or pushed in wheelchair. May be out |
| | of bed itself much of the day. Retains many self-care functions. Generally, has effective use of arms |
| 8.5 | Essentially restricted to bed much of day. Has some effective use of arms retains some self-care functions |
| 9.0 | Confined to bed. Can still communicate and eat |
| 9.5 | Confined to bed and totally dependent. Unable to communicate effectively or eat/swallow |
| 10.0 | Death due to MS |

Accordingly, an increase of the EDSS score over time indicates a progression of the disease, i.e., a worsening of the disability level. The absence of difference in the EDSS score over time indicates an absence of disease progression and a stable state of the MS patient. A small increase (or a positive difference) in the EDSS score indicates a limited progression of the disease. Thus, the smaller the increase (i.e., the smaller the positive difference), the more stable the MS patient. Accordingly, a smaller increase in the EDSS score, for example as compared to the progression of EDSS score over an identical time period before treatment, indicates a slowing down of the progression of the disease. A decrease in the EDSS score (or a negative difference) indicates an absence of disease progression and an improvement of the patient. Thus, the greater the decrease (i.e., the greater the negative difference), the greater the improvement.

In one embodiment, the object of the treatment is to bring about a decrease of the EDSS score of a patient suffering from progressive MS or to bring about a slowing down in the rate of increase of the EDSS score of a patient suffering from progressive MS.

Accordingly, in one embodiment, a patient suffering from progressive multiple sclerosis is considered as successfully “treated”, if, after receiving a therapeutically effective amount of a 2-aminoarylthiazole derivative as described herein, or a pharmaceutically acceptable salt or solvate thereof, the EDSS score of the patient has decreased or if the rate of increase of their EDSS score has slowed down.

According to one embodiment, disease progression in the patient suffering from progressive MS may be assessed with other tools commonly used in the field. Examples of such commonly used tools include, without being limited to, the Multiple Sclerosis Functional Composite (MSFC) and the Multiple Sclerosis Quality of Life (MSQOL-54).

In one embodiment, the patient is a male. In another embodiment, the patient is a female.

According to one embodiment, the patient is an adult. According to the present invention, an adult is a subject above the age of 18, 19, 20 or 21 years. In one embodiment, the patient is older than 20, 25, or 30 years.

According to one embodiment, the patient is a child. According to the present invention, a child is a subject below 21, 20, 19 or 18 years.

In one embodiment, the patient previously received a treatment for multiple sclerosis.

Examples of treatments for multiple sclerosis include, without being limited to, immunomodulators (such as immunomodulators as described herein), anti-inflammatory drugs, and nerve-targeting drugs (such as nerve-targeting drugs as described herein).

More particularly, examples of treatments for multiple sclerosis include, without being limited to, adrenocorticotropic hormone (ACTH), alemtuzumab, azathioprine, high-dose biotin (MD1003), cladribine, cyclophosphamide, cyclosporine, dalfampridine, dimethyl fumarate, diroximel fumarate, evobrutinib, fampridine, fingolimod, glatiramer acetate (also known as timexon or BCD-063), ibudilast, immunoglobulins, interferon (including interferon beta-1a (INF-β-1a), interferon beta-1b (INF-β-1b), peginterferon beta-1a, and peginterferon beta-1a biosimilar (also known as BCD-054)), laquinimod, methotrexate, mitoxantrone, mycophenolate mofetil, natalizumab, ocrelizumab, ofatumumab, ozanimod, ponesimod, simvastatin, siponimod, teriflunomide, and ublituximab.

Treatments for multiple sclerosis may also include, without being limited to, anti-inflammatory drugs such as non-steroidal anti-inflammatory drugs (NSAIDs), corticosteroids, analgesics such as analgesics without anti-inflammatory action or oral opioid analgesics.

In one embodiment, the patient previously received or has been receiving or is receiving a first-line treatment for multiple sclerosis, in particular for progressive multiple sclerosis. Thus, in one embodiment, the 2-aminoarylthiazole derivative as described herein, or a pharmaceutically acceptable salt or solvate thereof, is a further treatment for multiple sclerosis, in particular for progressive multiple sclerosis. In one embodiment, the 2-aminoarylthiazole derivative as described herein, or a pharmaceutically acceptable salt or solvate thereof, is a second-line treatment for multiple sclerosis, in particular for progressive multiple sclerosis.

In one embodiment, the 2-aminoarylthiazole derivative as described herein, or a pharmaceutically acceptable salt or solvate thereof, is to be administered as a further treatment, in particular in addition to a preexisting treatment, for multiple sclerosis, in particular for progressive multiple sclerosis. In one embodiment, the 2-aminoarylthiazole derivative as described herein, or a pharmaceutically acceptable salt or solvate thereof, is to be administered as a second-line treatment, in particular as a substitution for a first-line treatment, for multiple sclerosis, in particular for progressive multiple sclerosis.

In one embodiment, the patient failed to respond to a previously administered treatment for multiple sclerosis or showed irreversible progression of the disease despite previous administration of a treatment for multiple sclerosis. In one embodiment, the patient failed to respond to a previously administered first-line treatment for multiple sclerosis or showed irreversible progression of the disease despite previous administration of a first-line treatment for multiple sclerosis.

In one embodiment, the patient is resistant to a previously administered treatment for multiple sclerosis. In one embodiment, the patient is resistant to a previously administered first-line treatment for multiple sclerosis.

In the present invention, a 2-aminoarylthiazole derivative refers to a compound characterized by the presence of a thiazolyl group substituted on position 2 (i.e., between the heterocyclic nitrogen and sulfur atoms) by a secondary or tertiary amine, wherein the nitrogen atom of the amine is substituted by at least one aryl group.

According to one embodiment, the aryl group is substituted by an arylamide group (i.e., —NH—CO-aryl).

In one embodiment, the 2-aminoarylthiazole derivative of the invention has the following formula (I):

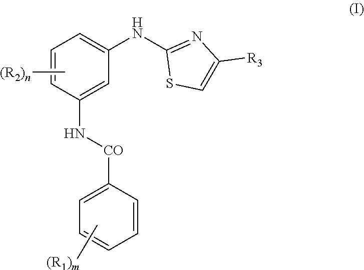

wherein:
R₁ and R₂ are selected independently from hydrogen, halogen, (C₁-C₁₀) alkyl, (C₃-C₁₀) cycloalkyl group, trifluoromethyl, alkoxy, cyano, dialkylamino, a solubilizing group, and (C₁-C₁₀) alkyl substituted by a solubilizing group;
m is 0-5;
n is 0-4;
R₃ is one of the following:
   (i) an aryl group (such as phenyl), the aryl group being optionally substituted by one or more substituents such as halogen, (C₁-C₁₀) alkyl group, trifluoromethyl, cyano and alkoxy;
   (ii) a heteroaryl group (such as 2, 3, or 4-pyridyl group), the heteroaryl group being optionally substituted by one or more substituents such as halogen, (C₁-C₁₀) alkyl group, trifluoromethyl and alkoxy;
   (iii) a five-membered ring aromatic heterocyclic group (such as, for example, 2-thienyl, 3-thienyl, 2-thiazolyl, 4-thiazolyl, 5-thiazolyl), the aromatic heterocyclic group being optionally substituted by one or more substituents such as halogen, (C₁-C₁₀) alkyl group, trifluoromethyl, and alkoxy.

In one embodiment, R₁ and R₂ of formula (I) are selected independently from hydrogen, halogen, (C₁-C₁₀) alkyl, (C₃-C₁₀) cycloalkyl group, trifluoromethyl, alkoxy, cyano, dialkylamino, and a solubilizing group.

Thus, in one embodiment, the 2-aminoarylthiazole derivative of the invention or a pharmaceutically acceptable salt or solvate thereof is a 2-aminoarylthiazole derivative of formula (I) or a pharmaceutically acceptable salt or solvate thereof.

In one embodiment, the 2-aminoarylthiazole derivative of the invention has the following formula (II):

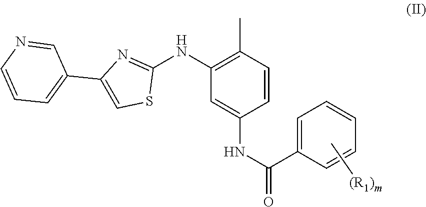

wherein:
R₁ is selected independently from hydrogen, halogen, (C₁-C₁₀) alkyl, (C₃-C₁₀) cycloalkyl group, trifluoromethyl, alkoxy, amino, alkylamino, dialkylamino, a solubilizing group, and (C₁-C₁₀) alkyl substituted by a solubilizing group; and m is 0-5.

In one embodiment, R₁ of formula (II) is selected independently from hydrogen, halogen, (C₁-C₁₀) alkyl, (C₃-C₁₀) cycloalkyl group, trifluoromethyl, alkoxy, amino, alkylamino, dialkylamino, and a solubilizing group.

In one embodiment, R₁ of formula (II) is a solubilizing group. In one embodiment, R₁ of formula (II) is (C₁-C₁₀) alkyl substituted by a solubilizing group.

In one embodiment, R₁ of formula (II) is (C₁-C₁₀) alkyl-(C₂-C₁₁) heterocycloalkyl-(C1-C10) alkyl-. In one embodiment, R₁ of formula (II) is (C₁-C₄) alkyl-(C₂-C₁₁) heterocyclo alkyl-(C₁-C₁₀) alkyl-, preferably (C₁-C₂) alkyl-(C₂-C₁₁) heterocycloalkyl-(C₁-C₁₀) alkyl-. In one embodiment, R₁ of formula (II) is (C₁-C₁₀) alkyl-(C₂-C₁₁) heterocycloalkyl-(C₁-C₄) alkyl-, preferably (C₁-C₁₀) alkyl-(C₂-C₁₁) heterocycloalkyl-(C₁-C₂) alkyl-. In one embodiment, R₁ of formula (II) is (C₁-C₁₀) alkyl-(C₂-C₆) heterocycloalkyl-(C₁-C₁₀) alkyl-, preferably (C₁-C₁₀) alkyl-(C₄) heterocycloalkyl-(C₁-C₁₀) alkyl-. In one embodiment, R₁ of formula (II) is (C₁-C₄) alkyl-(C₂-C₆) heterocycloalkyl-(C₁-C₄) alkyl-, preferably (C₁-C₂) alkyl-(C₄) heterocycloalkyl-(C₁-C₂) alkyl-. In one embodiment, R₁ of formula (II) is (C₁-C₄) alkyl-piperazinyl-(C₁-C₄) alkyl-, preferably (C₁-C₂) alkyl-piperazinyl-(C₁-C₂) alkyl-. In one embodiment, R₁ of formula (II) is methylpiperazinyl-(C₁-C₂) alkyl-, preferably methylpiperazinyl-methyl-, more preferably 4-methylpiperazinyl-methyl-.

Thus, in one embodiment, the 2-aminoarylthiazole derivative of the invention or a pharmaceutically acceptable salt or solvate thereof is a 2-aminoarylthiazole derivative of formula (II) as described above or a pharmaceutically acceptable salt or solvate thereof.

As used herein, the term “aryl group” refers to a polyunsaturated, aromatic hydrocarbyl group having a single aromatic ring (i.e., phenyl) or multiple aromatic rings fused together (e.g., naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Examples of suitable aryl groups include, without being limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C₆) aryl”.

As used herein, the term “alkyl group” refers to a saturated straight chain or branched non-cyclic hydrocarbon having from 1 to 10 carbon atoms, preferably from 1 to 6 carbon atoms. Representative saturated straight chain alkyls include, without being limited to, methyl, ethyl, n-propyl, n-butyl, n-pentyl, n-hexyl, n-heptyl, n-octyl, n-nonyl and n-decyl. Saturated branched alkyls include, without being limited to, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl. Alkyl groups included in compounds of the present invention may be optionally substituted with one or more substituents.

As used herein, the term “alkoxy” refers to an alkyl group which is attached to another moiety by an oxygen atom. Examples of alkoxy groups include, without being limited to, methoxy, isopropoxy, ethoxy, tert-butoxy. Alkoxy groups may be optionally substituted with one or more substituents.

As used herein, the term “cycloalkyl” refers to a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

As used herein, the term “halogen” refers to —F, —Cl, —Br or —I.

As used herein, the term “heteroaryl” refers to a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include, without being limited to, pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

As used herein, the term “heterocycle” refers collectively to heterocycloalkyl groups and heteroaryl groups.

As used herein, the term “heterocycloalkyl” refers to a monocyclic or polycyclic group having at least one heteroatom selected from O, N, or S, and which has 2-11 carbon atoms, which may be saturated or unsaturated, but is not aromatic. Examples of heterocycloalkyl groups include, without being limited to, piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein, the term “substituent” or “substituted” means that a hydrogen radical on a compound or group is replaced with any desired group that is substantially stable to reaction conditions in an unprotected form or when protected using a protecting group. Examples of preferred substituents include, without being limited to, halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an arylalkyl, a heteroarylalkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₁ and R₁₂ taken together with the nitrogen to which they are attached is optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In certain embodiments, the term “substituent” or the adjective “substituted” refers to a solubilizing group.

As used herein, the term “solubilizing group” refers to any group which can be substantially ionized and that enables the compound to be soluble in a desired solvent, such as, for example, water or water-containing solvent (“water-solubilizing group”). Furthermore, the solubilizing group can be one that increases the compound or complex's lipophilicity. In one embodiment, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. In one embodiment, the “solubilizing group” is one of the following:
an alkyl, cycloalkyl, aryl, heteroaryl group comprising either at least one nitrogen or oxygen heteroatom and/or which group is substituted by at least one amino group or oxo group (including, without being limited to, 2-oxopiperazinyl, 2-oxopiperidinyl, 2-oxopyrrolidinyl, 4-piperidonyl, hydantoinyl, valerolactamyl, oxiranyl, oxetanyl, tetrahydropyranyl, morpholinyl, 1,3-dioxolane, tetrahydrofuranyl and dihydrofuranyl-2-one);
an amino group which may be a saturated cyclic amino group (including, without being limited to, piperidinyl, piperazinyl and pyrrolidinyl) which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl (including, without being limited to, methyl-piperidinyl, methyl-piperazinyl and methyl-pyrrolidinyl);
one of the structures a) to i) shown below, wherein the wavy line and the arrow line correspond to the point of attachment to the core structure of the 2-aminoarylthiazole derivative of the invention, for example of formula (I) or (II):

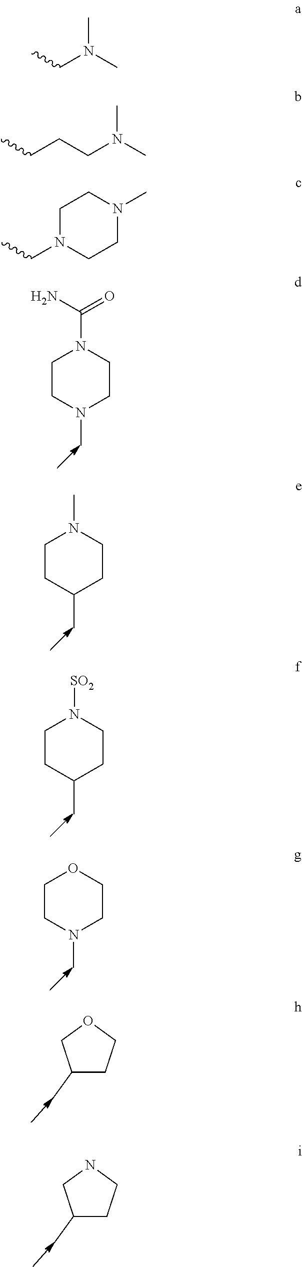

In one embodiment, the solubilizing group is a saturated cyclic amino group (including, without being limited to, piperidinyl, piperazinyl and pyrrolidinyl) which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl (including, without being limited to, methyl-piperidinyl, methyl-piperazinyl and methyl-pyrrolidinyl).

In one embodiment, the solubilizing group is structure c) shown above, wherein the wavy line corresponds to the point of attachment to the core structure of the 2-aminoarylthiazole derivative of the invention, for example of formula (I) or (II).

As used herein, “pharmaceutically acceptable salt” refers to a salt of a free acid or a free base which is not biologically undesirable and is generally prepared by reacting the free base with a suitable organic or inorganic acid or by reacting the free acid with a suitable organic or inorganic base. Suitable acid addition salts are formed from acids that form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, napsylate, nicotinate, nitrate, orotate, oxalate, palmitate, pamoate, phosphate/hydrogen, phosphate/dihydrogen, phosphate, pyroglutamate, saccharate, stearate, succinate, tannate, tartrate, tosylate, trifluoroacetate and xinofoate salts. Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine, 2 (diethylamino)ethanol, ethanolamine, morpholine, 4 (2 hydroxyethyl)morpholine and zinc salts. Hemi salts of acids and bases may also be formed, e.g., hemi sulphate and hemi calcium salts.

In one embodiment, pharmaceutically acceptable salts are pharmaceutically acceptable acid addition salts, for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid, or aromatic sulfonic acids, for example benzene-, p-toluene- or naphthalene-2-sulfonic acid.

In one embodiment, the pharmaceutically acceptable salt of the 2-aminoarylthiazole derivative of the invention is mesilate.

Unless otherwise indicated, the term “mesilate” is used herein to refer to a salt of methanesulfonic acid with a named pharmaceutical substance (such as compounds of formula (I) or (II)). Use of mesilate rather than mesylate is in compliance with the INNM (International nonproprietary names modified) issued by WHO (e.g., World Health Organization (February 2006). International Nonproprietary Names Modified. INN Working Document 05.167/3. WHO).

As used herein, “pharmaceutically acceptable solvate” refers to a molecular complex comprising the 2-aminoarylthiazole derivative of the invention and stoichiometric or sub-stoichiometric amounts of one or more pharmaceutically acceptable solvent molecules such as ethanol. The term ‘hydrate’ refers to when said solvent is water.

In one embodiment, the 2-aminoarylthiazole derivative of the invention or a pharmaceutically acceptable salt or solvate thereof is masitinib or a pharmaceutically acceptable salt or solvate thereof.

The chemical name for masitinib is 4-(4-methylpiperazin-1-ylmethyl)-N-[4-methyl-3-(4-pyridin-3ylthiazol-2-ylamino) phenyl]benzamide—CAS number 790299-79-5:

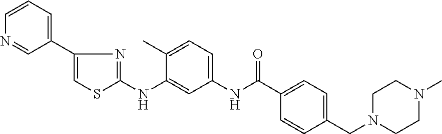 [SMILES: Cc1ccc(NC(=O)c2ccc(CN3CCN(C)CC3)cc2)cc1Nc1nc(-c2cccnc2)cs1]

Masitinib was first described in U.S. Pat. No. 7,423,055 and EP 1 525 200 B1.

In one embodiment, the 2-aminoarylthiazole derivative of the invention or a pharmaceutically acceptable salt or solvate thereof is masitinib mesilate. Thus, in one embodiment, the pharmaceutically acceptable salt of masitinib as described hereinabove is masitinib mesilate. As mentioned hereinabove, in other words, the pharmaceutically acceptable salt of masitinib is the methanesulfonic acid salt of masitinib.

A detailed procedure for the synthesis of masitinib mesilate is given in WO 2008/098949.

In one embodiment, “masitinib mesilate” refers to the orally bioavailable mesilate salt of masitinib—CAS 1048007-93-7 (MsOH); C₂₈H₃₀N₆OS.CH₃SO₃H; MW 594.76:

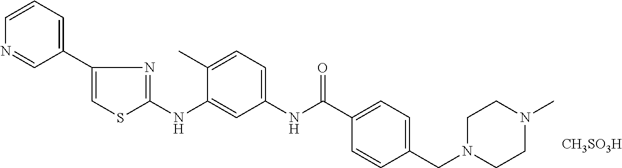

According to one embodiment, the 2-aminoarylthiazole derivative as described hereinabove, in particular masitinib, or a pharmaceutically acceptable salt or solvate thereof, is to be administered at a therapeutically effective dose.

In one embodiment, the 2-aminoarylthiazole derivative as described hereinabove, in particular masitinib, or a pharmaceutically acceptable salt or solvate thereof, is to be administered at a dose ranging from about 1 to about 12 mg/kg/day (mg per kilo body weight per day). In one embodiment, the 2-aminoarylthiazole derivative as described hereinabove, in particular masitinib, is to be administered at a dose ranging from about 1.5 to about 7.5 mg/kg/day. In one embodiment, the 2-aminoarylthiazole derivative as described hereinabove, in particular masitinib, or a pharmaceutically acceptable salt or solvate thereof, is to be administered at a dose ranging from about 3 to about 12 mg/kg/day, preferably from about 3 to about 6 mg/kg/day.

In one embodiment, the 2-aminoarylthiazole derivative as described hereinabove, in particular masitinib, is to be administered at a dose of about 1, 2, 3, 4, 5, 6, 7, 8, 9, 10, 11 or 12 mg/kg/day. In one embodiment, the 2-aminoarylthiazole derivative as described hereinabove, in particular masitinib, or a pharmaceutically acceptable salt or solvate thereof, is to be administered at a dose of about 1.5, 3, 4.5, 6, 7.5, 9, 10.5 or 12 mg/kg/day.

In one embodiment, the 2-aminoarylthiazole derivative as described hereinabove, in particular masitinib, or a pharmaceutically acceptable salt or solvate thereof, is to be administered at a dose of about 3, 4.5 or 6 mg/kg/day, preferably at a dose of about 4.5 mg/kg/day.

In one embodiment, the 2-aminoarylthiazole derivative as described hereinabove, in particular masitinib, or a pharmaceutically acceptable salt or solvate thereof, can be dose escalated by increments of about 1.5 mg/kg/day to reach a maximum of about 12 mg/kg/day, more preferably of about 7.5 mg/kg/day, even more preferably of about 6 mg/kg/day. Each dose escalation is subjected to toxicity controls with an absence of any toxicity events permitting dose escalation to occur.

In one embodiment, the dose escalation of the 2-aminoarylthiazole derivative, or a pharmaceutically acceptable salt or solvate thereof, occurs at any time-point after at least 4 weeks after the administration of the initial dose and prior to 26 weeks after the administration of the initial dose; for example at 4 weeks, 8 weeks, 12 weeks, 16 weeks, 20 weeks, or 24 weeks after the administration of the initial dose. Each dose escalation is subjected to toxicity controls. Examples of a toxicity control include assessing that, during the previous 4-week treatment period at a constant dose of study treatment, no suspected severe adverse event was reported, no suspected adverse event led to treatment interruption, and/or no suspected adverse event is ongoing at the time of the dose increase, regardless of its severity.

In one embodiment, the 2-aminoarylthiazole derivative as described hereinabove, in particular masitinib, or a pharmaceutically acceptable salt or solvate thereof, is to be administered at an initial dose of about 3 mg/kg/day during at least 6 weeks, at least 9 weeks, or at least 12 weeks, then at a dose of about 4.5 mg/kg/day thereafter.

In one embodiment, the 2-aminoarylthiazole derivative as described hereinabove, in particular masitinib, or a pharmaceutically acceptable salt or solvate thereof, is to be administered at an initial dose of about 3 mg/kg/day during at least 4 weeks, then at a dose of about 4.5 mg/kg/day during at least 4 weeks, and at a dose of about 6 mg/kg/day thereafter, with each dose escalation being subjected to toxicity controls.

In one embodiment, the 2-aminoarylthiazole derivative as described hereinabove, in particular masitinib, or a pharmaceutically acceptable salt or solvate thereof, is to be administered at an initial dose of about 4.5 mg/kg/day during at least 6 weeks, at least 9 weeks, or at least 12 weeks, and at a dose of about 6 mg/kg/day thereafter, with each dose escalation being subjected to toxicity controls. In one embodiment, the 2-aminoarylthiazole derivative as described hereinabove, in particular masitinib, or a pharmaceutically acceptable salt or solvate thereof, is to be administered at an initial dose of about 4.5 mg/kg/day during 12 weeks, and at a dose of about 6 mg/kg/day thereafter, with each dose escalation being subjected to toxicity controls.

According to one embodiment, any dose indicated herein refers to the amount of active ingredient as such, not to its pharmaceutically acceptable salt or solvate form. Thus, compositional variations of a pharmaceutically acceptable salt or solvate of the 2-aminoarylthiazole derivative of the invention, in particular masitinib, will not impact the dose to be administered.

According to one embodiment, the 2-aminoarylthiazole derivative as described hereinabove, in particular masitinib, or a pharmaceutically acceptable salt or solvate thereof, may be administered orally, intravenously, parenterally, topically, by inhalation spray, rectally, nasally, or buccally.

In one embodiment, the 2-aminoarylthiazole derivative as described hereinabove, in particular masitinib, or a pharmaceutically acceptable salt or solvate thereof, is to be administered orally.

In one embodiment, the 2-aminoarylthiazole derivative as described hereinabove, in particular masitinib, or a pharmaceutically acceptable salt or solvate thereof, is to be administered at least once a day, preferably twice a day.

In one embodiment, the 2-aminoarylthiazole derivative as described hereinabove, in particular masitinib, or a pharmaceutically acceptable salt or solvate thereof, is to be administered for a prolonged period, such as for example, for at least 1, 2, 3, 6, 9, or 12 months.

In one embodiment, the 2-aminoarylthiazole derivative as described hereinabove, in particular masitinib, or a pharmaceutically acceptable salt or solvate thereof, is in a form adapted for oral administration.

Examples of forms adapted for oral administration include, without being limited to, liquid, paste or solid compositions, and more particularly tablets, pills, capsules, liquids, gels, syrups, slurries, and suspensions.

In one embodiment, the 2-aminoarylthiazole derivative as described hereinabove, in particular masitinib, or a pharmaceutically acceptable salt or solvate thereof, is to be administered as tablets, preferably as 100 mg or 200 mg tablets.

According to one embodiment, the 2-aminoarylthiazole derivative as described hereinabove, in particular masitinib, or a pharmaceutically acceptable salt or solvate thereof, is for administration as a further or second-line treatment and/or as an add-on to supportive care or to palliative care.

In one embodiment, the 2-aminoarylthiazole derivative as described hereinabove, in particular masitinib, or a pharmaceutically acceptable salt or solvate thereof, is for administration as a further or second-line treatment.

In one embodiment, the 2-aminoarylthiazole derivative as described hereinabove, in particular masitinib, or a pharmaceutically acceptable salt or solvate thereof, is for administration as an add-on to supportive care or to palliative care.

According to one embodiment, the 2-aminoarylthiazole derivative as described hereinabove, in particular masitinib, or a pharmaceutically acceptable salt or solvate thereof, is to be administered with at least another pharmaceutically active agent.

According to the present invention, the 2-aminoarylthiazole derivative as described hereinabove, in particular masitinib, or a pharmaceutically acceptable salt or solvate thereof, may be administered simultaneously, separately or sequentially with said at least another pharmaceutically active agent.

In one embodiment, the 2-aminoarylthiazole derivative as described hereinabove, in particular masitinib, or a pharmaceutically acceptable salt or solvate thereof, is to be administered in combination with said at least another pharmaceutically active agent, such as in a combined preparation, pharmaceutical composition or medicament.

In one embodiment, the 2-aminoarylthiazole derivative as described hereinabove, in particular masitinib, or a pharmaceutically acceptable salt or solvate thereof, and the at least another pharmaceutically active agent are to be administered separately.

Examples of other pharmaceutically active agents that may be administered to the patient with progressive MS include, without being limited to, immunomodulators, anti-inflammatory drugs, and nerve-targeting drugs.

In one embodiment, the at least another pharmaceutically active agent is selected from the group comprising or consisting of immunomodulators, anti-inflammatory drugs, and nerve-targeting drugs.

In one embodiment, the at least another pharmaceutically active agent is selected from the group comprising or consisting of adrenocorticotropic hormone (ACTH), alemtuzumab, azathioprine, high-dose biotin (MD1003), cladribine, cyclophosphamide, cyclosporine, dalfampridine, dimethyl fumarate, diroximel fumarate, evobrutinib, fampridine, fingolimod, glatiramer acetate (also known as timexon or BCD-063), ibudilast, immunoglobulins, interferon (including interferon beta-1a (INF-β-1a), interferon beta-1b (INF-β-1b), peginterferon beta-1a, and peginterferon beta-1a biosimilar (also known as BCD-054)), laquinimod, methotrexate, mitoxantrone, mycophenolate mofetil, natalizumab, ocrelizumab, ofatumumab, ozanimod, ponesimod, simvastatin, siponimod, teriflunomide, and ublituximab.

In one embodiment, the at least another pharmaceutically active agent is an immunomodulator, i.e., an agent modulating an immune response. As used herein, immunomodulators include both agents suppressing an immune response and agents stimulating an immune response. Examples of immunomodulators include, without being limited to, adrenocorticotropic hormone (ACTH), alemtuzumab, azathioprine, cladribine, cyclophosphamide, cyclosporine, dimethyl fumarate, diroximel fumarate, evobrutinib, fingolimod, glatiramer acetate (also known as timexon or BCD-063), immunoglobulins, interferon (including interferon beta-1a (INF-β-1a), interferon beta-1b (INF-β-1b), peginterferon beta-1a, and peginterferon beta-1a biosimilar (also known as BCD-054)), laquinimod, methotrexate, mitoxantrone, mycophenolate mofetil, natalizumab, ocrelizumab, ofatumumab, ozanimod, ponesimod, simvastatin, siponimod, teriflunomide, and ublituximab. Examples of immunosuppressants include, without being limited to, alemtuzumab, azathioprine, cladribine, cyclophosphamide, cyclosporine, dimethyl fumarate, fingolimod, laquinimod, methotrexate, mitoxantrone, mycophenolate mofetil, natalizumab, ocrelizumab, ofatumumab, ozanimod, ponesimod, siponimod, teriflunomide, and ublituximab.

In one embodiment, the at least another pharmaceutically active agent is an anti-inflammatory drug. Examples of anti-inflammatory drugs include, without being limited, adrenocorticotropic hormone (ACTH), ibudilast, and immunoglobulins.

In one embodiment, the at least another pharmaceutically active agent is a nerve-targeting drug. As used herein, examples of nerve-targeting drugs include, without being limited to, high-dose biotin (MD1003), dalfampridine, and fampridine.

In one embodiment, the at least another pharmaceutically active agent is selected from the group comprising or consisting of high-dose biotin (MD1003), ibudilast, ocrelizumab, simvastatin, and siponimod.

According to one embodiment, the 2-aminoarylthiazole derivative as described hereinabove, in particular masitinib, or a pharmaceutically acceptable salt or solvate thereof, optionally with the at least another pharmaceutically active agent as described hereinabove, is to be administered with at least one further pharmaceutically active agent aiming at treating one or more symptoms or manifestations of MS. Examples of further pharmaceutically active agents aiming at treating one or more symptoms of MS include, without being limited to, anti-inflammatory drugs such as non-steroidal anti-inflammatory drugs (NSAIDs), corticosteroids, analgesics such as analgesics without anti-inflammatory action or oral opioid analgesics, muscle relaxants, medications to reduce fatigue, antidepressants, antimuscarinics, and laxatives.

In one embodiment, the at least one further pharmaceutically active agent is selected from the group comprising or consisting of anti-inflammatory drugs such as non-steroidal anti-inflammatory drugs (NSAIDs), corticosteroids, analgesics such as analgesics without anti-inflammatory action or oral opioid analgesics.

In one embodiment, the at least one further pharmaceutically active agent is an anti-inflammatory drug, in particular a non-steroidal anti-inflammatory drug (NSAID). Examples of NSAIDs include, without being limited to, ibuprofen and naproxen.

In one embodiment, the at least one further pharmaceutically active agent is a corticosteroid. Examples of corticosteroids include, without being limited to, methylprednisolone, dexamethasone, prednisone, prednisolone, and betamethasone.

In one embodiment, the at least one further pharmaceutically active agent is an analgesic, such as analgesics an analgesic without anti-inflammatory action or an oral opioid analgesic. Examples of analgesics without anti-inflammatory action include, without being limited to, acetaminophen. Examples of oral opioid analgesics include, without being limited to, morphine, oxycodone, and methadone.

In one embodiment, the 2-aminoarylthiazole derivative as described hereinabove, in particular masitinib, or a pharmaceutically acceptable salt or solvate thereof, is to be administered with at least one pharmaceutically active agent selected from the group comprising or consisting of non-steroidal anti-inflammatory drugs (NSAIDs), corticosteroids, analgesics without anti-inflammatory action, oral opioid analgesics, adrenocorticotropic hormone (ACTH), alemtuzumab, azathioprine, high-dose biotin (MD1003), cladribine, cyclophosphamide, cyclosporine, dalfampridine, dimethyl fumarate, diroximel fumarate, evobrutinib, fampridine, fingolimod, glatiramer acetate (also known as timexon or BCD-063), ibudilast, immunoglobulins, interferon (including interferon beta-1a (INF-β-1a), interferon beta-1b (INF-β-1b), peginterferon beta-1a, and peginterferon beta-1a biosimilar (also known as BCD-054)), laquinimod, methotrexate, mitoxantrone, mycophenolate mofetil, natalizumab, ocrelizumab, ofatumumab, ozanimod, ponesimod, simvastatin, siponimod, teriflunomide, and ublituximab.

In one embodiment, the 2-aminoarylthiazole derivative as described hereinabove, in particular masitinib, or a pharmaceutically acceptable salt or solvate thereof, is to be administered with at least one pharmaceutically active agent selected from the group comprising or consisting of non-steroidal anti-inflammatory drugs (NSAIDs), corticosteroids, analgesics without anti-inflammatory action, oral opioid analgesics, high-dose biotin (MD1003), ibudilast, ocrelizumab, simvastatin, and siponimod.

Another object of the present invention is a method for treating progressive multiple sclerosis (MS) in a patient in need thereof, comprising administering to the patient a 2-aminoarylthiazole derivative as described hereinabove, in particular masitinib, or a pharmaceutically acceptable salt or solvate thereof, wherein said patient has a time from diagnosis to treatment initiation with the 2-aminoarylthiazole derivative, or a pharmaceutically acceptable salt or solvate thereof, greater than 1 year, preferably greater than 2 years and/or has a time from onset to treatment initiation with the 2-aminoarylthiazole derivative, or a pharmaceutically acceptable salt or solvate thereof, greater than 2 years, preferably greater than 5 years.

In one embodiment, the patient has a time from diagnosis to treatment initiation with the 2-aminoarylthiazole derivative, or a pharmaceutically acceptable salt or solvate thereof, greater than 3 years, and/or has a time from onset to treatment initiation with the 2-aminoarylthiazole derivative, or a pharmaceutically acceptable salt or solvate thereof, greater than 5 years.

The present invention also relates to a method for treating progressive multiple sclerosis (MS) in a patient in need thereof, comprising administering to the patient a 2-aminoarylthiazole derivative as described hereinabove, in particular masitinib, or a pharmaceutically acceptable salt or solvate thereof, optionally as a further or second-line treatment and/or as an add-on to supportive care or to palliative care, 1 year or more, preferably 2 years or more, from diagnosis (i.e., from the time of diagnosis) and/or 2 years or more, preferably 5 years or more, from disease onset (i.e., from the time of disease onset).

The present invention also relates to a method for treating progressive multiple sclerosis (MS) in a patient in need thereof, comprising administering to the patient a 2-aminoarylthiazole derivative as described hereinabove, in particular masitinib, or a pharmaceutically acceptable salt or solvate thereof, optionally as a further or second-line treatment and/or as an add-on to supportive care or to palliative care, 3 years or more from diagnosis (i.e., from the time of diagnosis) and/or 5 years or more from disease onset (i.e., from the time of disease onset).

Another object of the present invention is a pharmaceutical composition for use in the treatment of progressive MS or for treating progressive MS in a patient in need thereof, wherein said pharmaceutical composition comprises a 2-aminoarylthiazole derivative as described hereinabove, in particular masitinib, or a pharmaceutically acceptable salt or solvate thereof, and wherein said patient has a time from diagnosis to treatment initiation with the 2-aminoarylthiazole derivative, or a pharmaceutically acceptable salt or solvate thereof, greater than 1 year, preferably greater than 2 years and/or has a time from onset to treatment initiation with the 2-aminoarylthiazole derivative, or a pharmaceutically acceptable salt or solvate thereof, greater than 2 years, preferably greater than 5 years.

In one embodiment, the present invention relates to a pharmaceutical composition for use in the treatment of progressive MS or for treating progressive MS in a patient in need thereof, wherein said pharmaceutical composition comprises a 2-aminoarylthiazole derivative as described hereinabove, in particular masitinib, or a pharmaceutically acceptable salt or solvate thereof, and wherein said patient has a time from diagnosis to treatment initiation with the 2-aminoarylthiazole derivative, or a pharmaceutically acceptable salt or solvate thereof, greater than 3 years and/or has a time from onset to treatment initiation with the 2-aminoarylthiazole derivative, or a pharmaceutically acceptable salt or solvate thereof, greater than 5 years.

In one embodiment, the pharmaceutical composition as described hereinabove comprises at least one pharmaceutically acceptable excipient.

The present invention also relates to a pharmaceutical composition for use in the treatment of progressive MS or for treating progressive MS in a patient in need thereof, wherein said pharmaceutical composition comprises a 2-aminoarylthiazole derivative as described hereinabove, in particular masitinib, or a pharmaceutically acceptable salt or solvate thereof, and wherein said pharmaceutical composition is for administration, optionally as a further or second-line treatment and/or as an add-on to supportive care or to palliative care, 1 year or more, preferably 2 years or more, from diagnosis (i.e., from the time of diagnosis) and/or 2 years or more, preferably 5 years or more, from disease onset (i.e., from the time of disease onset).

In one embodiment, the present invention relates to a pharmaceutical composition for use in the treatment of progressive MS or for treating progressive MS in a patient in need thereof, wherein said pharmaceutical composition comprises a 2-aminoarylthiazole derivative as described hereinabove, in particular masitinib, or a pharmaceutically acceptable salt or solvate thereof, and wherein said pharmaceutical composition is for administration, optionally as a further or second-line treatment and/or as an add-on to supportive care or to palliative care, 3 years or more from diagnosis (i.e., from the time of diagnosis) and/or 5 years or more from disease onset (i.e., from the time of disease onset).

Another object of the present invention is a 2-aminoarylthiazole derivative as described hereinabove, in particular masitinib, or a pharmaceutically acceptable salt or solvate thereof, for use in the manufacture of a medicament for the treatment of progressive MS in a patient in need thereof, wherein said patient has a time from diagnosis to treatment initiation with the 2-aminoarylthiazole derivative, or a pharmaceutically acceptable salt or solvate thereof, greater than 1 year, preferably greater than 2 years and/or has a time from onset to treatment initiation with the 2-aminoarylthiazole derivative, or a pharmaceutically acceptable salt or solvate thereof, greater than 2 years, preferably greater than 5 years.

In one embodiment, the present invention relates to a 2-aminoarylthiazole derivative as described hereinabove, in particular masitinib, or a pharmaceutically acceptable salt or solvate thereof, for use in the manufacture of a medicament for the treatment of progressive MS in a patient in need thereof, wherein said patient has a time from diagnosis to treatment initiation with the 2-aminoarylthiazole derivative, or a pharmaceutically acceptable salt or solvate thereof, greater than 3 years and/or has a time from onset to treatment initiation with the 2-aminoarylthiazole derivative, or a pharmaceutically acceptable salt or solvate thereof, greater than 5 years.

The present invention also relates to a 2-aminoarylthiazole derivative as described hereinabove, in particular masitinib, or a pharmaceutically acceptable salt or solvate thereof, for use in the manufacture of a medicament for the treatment of progressive MS in a patient in need thereof, wherein said medicament is for administration, optionally as a further or second-line treatment and/or as an add-on to supportive care or to palliative care, 1 year or more, preferably 2 years or more, from diagnosis (i.e., from the time of diagnosis) and/or 2 years or more, preferably 5 years or more, from disease onset (i.e., from the time of disease onset).

In one embodiment, the present invention relates to a 2-aminoarylthiazole derivative as described hereinabove, in particular masitinib, or a pharmaceutically acceptable salt or solvate thereof, for use in the manufacture of a medicament for the treatment of progressive MS in a patient in need thereof, wherein said medicament is for administration, optionally as a further or second-line treatment and/or as an add-on to supportive care or to palliative care, 3 years or more from diagnosis (i.e., from the time of diagnosis) and/or 5 years or more from disease onset (i.e., from the time of disease onset).

The present invention also relates to a 2-aminoarylthiazole derivative as described hereinabove, in particular masitinib, or a pharmaceutically acceptable salt or solvate thereof, for use in the manufacture of a medicament for the treatment of late-phase progressive MS (i.e., the late phase of progressive MS) in a patient in need thereof, wherein late-phase progressive MS is defined as a time since diagnosis of 1 year or more, preferably 2 years or more, and/or as a time since disease onset of 2 years or more, preferably 5 years or more. In other words, the present invention also relates to a 2-aminoarylthiazole derivative as described hereinabove, in particular masitinib, or a pharmaceutically acceptable salt or solvate thereof, for use in the manufacture of a medicament for the treatment of late-phase progressive MS (i.e., the late phase of progressive MS) in a patient in need thereof, wherein late-phase progressive MS is defined as a time from diagnosis of at least 1 year, preferably at least 2 years, and/or as a time from disease onset of at least 2 years, preferably at least 5 years.

In one embodiment, late-phase progressive MS is defined as a time since diagnosis of 3 years or more and/or as a time since disease onset of 5 years or more. In other words, in one embodiment, late-phase progressive MS is defined as a time from diagnosis of at least 3 years and/or as a time from disease onset of at least 5 years.

### EXAMPLES

The present invention is further illustrated by the following examples.

### Example 1

### Study AB07002 Design and Primary Analysis Results

Methods

An international, multicenter, randomized, double-blind, placebo-controlled phase 3 study was conducted with the objective to evaluate oral masitinib at a dose of 4.5 mg/kg/day as a treatment for progressive multiple sclerosis (progressive MS), as defined hereinabove. Eligible patients, aged 18-75 years, were diagnosed with primary progressive multiple sclerosis (PPMS) or non-active secondary progressive multiple sclerosis (non-active SPMS or nSPMS), and had a baseline score on the Expanded Disability Status Scale (EDSS) ranging from 2.0 to 6.0. Eligible patients were treated for 96 weeks with masitinib at a dose of 4.5 mg/kg/day (mg per kilo body weight per day) or with placebo.

Primary endpoint was overall EDSS change from baseline using a repeated analysis of covariance model (i.e., ANCOVA GEE model on all measurements from week 12 (W12) to week 96 (W96)).

Results are expressed as least-squares means difference, illustrating the progression of EDSS score, that is to say the change over time in the EDSS score or in other words the difference between the EDSS score at baseline and the EDSS score after treatment with masitinib or with placebo (a positive value indicates a worsening of the patient). Treatment-effect is reported as between-group difference (ΔLSM, negative value indicates a beneficial effect of treatment with masitinib as compared to treatment with placebo).

Inclusion criteria included:
Patients suffering from either primary progressive MS (PPMS) or secondary progressive multiple sclerosis without relapse within 2 years before inclusion according to the revised McDonald criteria (non-active SPMS or nSPMS);
Patients with EDSS score ranging from 2.0 to 6.0 inclusive at baseline;
Patients who had an EDSS score progression≥1 point within 2 years before inclusion; and
Male or female patients aged between 18 and 75 years old, with a weight>50 kg and (body mass index) BMI between 18 and 35 kg/m².

Patients suffering from a disease other than MS that would better explain their neurological clinical signs and symptoms and/or lesions observed with magnetic resonance imaging (“MRI lesions”) were excluded.

The revised McDonald criteria for the diagnosis of MS is a well-established diagnostic instrument that is widely used in research and clinical practice. The McDonald criteria for the diagnosis of multiple sclerosis have been published in Polman et al., Ann Neurol. 2011; 69(2):292-302. The 2017 revisions of the McDonald criteria have been published in Thompson et al., Lancet Neurol. 2018; 17(2):162-173. Anyone who was diagnosed using previous versions of the McDonald Criteria will also meet the criteria for MS as laid out in the 2017 McDonald Criteria.

The term “secondary progressive” illustrates the fact that secondary progressive MS (SPMS) is diagnosed in a patient who has previously experienced RRMS. A patient is considered to have transitioned to a SPMS disease course when disease is continuing to worsen even though the MS patient is no longer experiencing inflammatory relapses.

Results

Table 4 below presents the primary efficacy analysis results from the masitinib 4.5 mg/kg/day full dataset of study AB07002 with breakdown of the progressive MS population into its component PPMS and non-active SPMS subgroups. In brief:
Masitinib demonstrated efficacy with respect to placebo for treatment of progressive MS, as evidenced by significant slowing of EDSS progression (see LSM EDSS after treatment with masitinib as compared to LSM EDSS after treatment with placebo, or see AEDSS);
Masitinib demonstrated a non-significant trend with respect to placebo for treatment of PPMS, as evidenced by a slowing of EDSS progression (see LSM EDSS after treatment with masitinib as compared to LSM EDSS after treatment with placebo, or see AEDSS);
Masitinib demonstrated a significant slowing of EDSS progression with respect to placebo for treatment of non-active SPMS; indeed, on average, there was an apparent stop of EDSS progression (see the negative value of LSM EDSS after treatment with masitinib, indicating an EDSS score after treatment with masitinib lower than the EDSS score at baseline).

TABLE 4
Comparison of masitinib- versus placebo-treated
patients in the full analysis dataset

| | N | | LSM EDSS | | | |
|---|---|---|---|---|---|---|
| | MAS | PBO | MAS | PBO | ΔEDSS [CI] | P-value |
| Progressive | 199 | 101 | 0.008 | 0.107 | −0.099 | 0.022 |
| MS | | | | | [−0.183, −0.014] | |
| PPMS | 79 | 45 | 0.030 | 0.163 | −0.133 | 0.1021 |
| | | | | | [−0.292, 0.0264] | |
| nSPMS | 120 | 56 | −0.035 | 0.059 | −0.095 | 0.0348 |
| | | | | | [−0.182, −0.006] | |

MAS: Masitinib. PBO: Placebo. LSM: Least squares means. EDSS: Expanded Disability Status Scale. ΔEDSS: Between group difference (treatment-effect). PPMS: primary progressive MS. nSPMS: non-active secondary progressive MS.

### Example 2

### Analysis of Progressive MS Subpopulations According to the Combined Clinical Markers ‘Time from Diagnosis’ and ‘Time from Onset’

Methods

Subpopulation analyses of primary endpoint data from study AB07002 assessed the impact of the clinical markers ‘time from onset’ with respect to baseline (i.e., date of treatment initiation), derived from the estimated date of first clinical MS symptom(s), or ‘time from diagnosis’ with respect to baseline (i.e., date of treatment initiation), on masitinib efficacy in patients with progressive MS (see Example 3).

Results surprisingly show that only the patients receiving treatment at a later phase of the disease development (i.e., at least 1 year following MS diagnosis and/or at least 2 years following first symptom(s) of disease) are susceptible to benefit from masitinib treatment. Patients in an earlier phase of progressive MS disease development (i.e., soon after MS diagnosis or first symptom(s) of disease) do not display a discernable response to masitinib as compared with placebo. Hence, the clinical markers of ‘time from diagnosis to treatment initiation’ and ‘time from onset to treatment initiation’ can be used as predictive indicators of likely response to masitinib and therefore as an instrument for selection of patients most likely to benefit from masitinib treatment.

‘Time from onset’ is derived from the date (year/month/day, year/month, or year) of first clinical symptom(s) with duration calculated from date of treatment initiation. The date of first symptom(s) is typically determined retrospectively from medical history, for example from medical records and patient/caregiver historical recall of MS related symptoms following a clinically definite diagnosis. Imputation of missing data can be handled using various methods, one example of which is the following approach: if the information regarding day is missing, the day is considered to be the last day of the month; if the day and month are missing, the day is considered to be the last day of June.

‘Time from diagnosis’ of progressive MS, is ideally based on a clinically definite diagnosis of progressive MS as determined by a MS specialist neurologist. In practice it is derived from the date (year/month/day, year/month, or year) of first clinically definite diagnosis of progressive MS, i.e., PPMS or non-active SPMS, with duration calculated from date of treatment initiation. At the time of MS diagnosis, a provisional disease course should be specified (relapsing-remitting, primary progressive, or secondary progressive) and whether the course is active or not (non-active) and progressive or not based on the previous year's history. The phenotype should be periodically re-evaluated based on accumulated information.

The initial diagnosis can be either relapse-remitting at onset (RRMS) or progressive at onset (PPMS). In general, the process of diagnosis involves obtaining evidence from a clinical examination, medical history, laboratory tests and magnetic resonance imaging (MRI) of the brain and sometimes the spinal cord. These tests are intended to rule out other possible causes of a person's neurological symptoms and to gather data consistent with MS. In the absence of a validated biological marker, clinically definite diagnosis of MS is made only after demonstrating objective clinical evidence of central nervous system (CNS) involvement with spatial dissemination of lesions (suggesting damage in more than one place in the nervous system) and temporal dissemination of lesions (suggesting that damage has occurred more than once). These key principles are embodied by the 2017 revised McDonald criteria for the diagnosis of multiple sclerosis (Thompson et al., Lancet Neurol. 2018; 17(2):162-173), an instrument that is widely used in research and clinical practice (see hereinabove for further details). Anyone who was diagnosed using previous versions of the McDonald criteria will also meet the criteria for MS as described in the 2017 McDonald criteria. A patient is considered to have transitioned from the RRMS disease course to the non-active secondary-progressive MS (SPMS) disease course when disease is continuing to worsen even though the patient is no longer experiencing inflammatory relapses. A variety of strategies can be employed to make a diagnosis of SPMS, including a careful history of the changes in a person's symptoms, the neurologic examination, and repeat MRI scans.

Results

More specifically, the abovementioned clinical data show that orally administered masitinib (4.5 mg/kg/day) provides greater therapeutic benefit to a distinct subpopulation of the ‘progressive MS’ population (see Table 5 below). This enhanced treatment-effect, as measured by change on EDSS score, is in terms of a uniform (i.e., progressive MS population and its component subgroups of PPMS and non-active SPMS) statistically significant difference between masitinib and placebo treatment-arms, as well as actual decrease in disability with respect to baseline, as evidenced by a uniform reversal (i.e., decline) in overall EDSS scores, that is not seen in the placebo treatment-arm.

Comparison of subpopulation analyses with respect to the corresponding full analysis dataset (see Table 4) is reported as relative difference in ΔEDSS (R %). A large positive R % value indicates a subpopulation that receives greater benefit from masitinib treatment, while a negative R % value indicates a diminished treatment-effect or lack of discernable treatment-effect in the subpopulation.

TABLE 5
Comparison of masitinib- versus placebo-treated patients in subpopulations
defined using the clinical markers of time from diagnosis to treatment
initiation and time from onset to treatment initiation

| N | | LSM EDSS | | | | |
|---|---|---|---|---|---|---|
| MAS | PBO | MAS | PBO | ΔEDSS (95% CI) | P-value | R % |

Progressive MS

| | | | | | | | |
|---|---|---|---|---|---|---|---|
| T (diag) > 2 Y | 152 | 73 | −0.057 | 0.105 | −0.162 | 0.0003 | 64% |
| T(onset) > 5 Y | | | | | (−0.249, −0.074) | | |
| T(diag) ≤ 2 Y | 47 | 28 | 0.173 | 0.035 | 0.139 | | |
| T(onset) ≤ 5 Y | | | | | (−0.057, 0.3344) | | |

PPMS

| | | | | | | | |
|---|---|---|---|---|---|---|---|
| T(diag) > 2 Y | 39 | 20 | −0.076 | 0.207 | −0.283 | 0.0031 | 113% |
| T(onset) > 5 Y | | | | | (−0.470, 0.095) | | |
| T(diag) ≤ 2 Y | 40 | 25 | 0.180 | 0.062 | 0.118 | | |
| T(onset) ≤ 5 Y | | | | | (−0.133, 0.3693) | | |

nSPMS

| | | | | | | | |
|---|---|---|---|---|---|---|---|
| T(diag) > 2 Y | 113 | 53 | −0.051 | 0.067 | −0.118 | 0.0094 | 24% |
| T(onset) > 5 Y | | | | | (−0.207, −0.028) | | |
| T(diag) ≤ 2 Y | 7 | 3 | N/A | N/A | N/A | | |
| T(onset) ≤ 5 Y | | | | | | | |

MAS: Masitinib. PBO: Placebo. LSM: Least squares means. EDSS: Expanded Disability Status Scale. ΔEDSS: Between group difference (treatment-effect). R %: relative difference in ΔEDSS with respect to full analysis dataset. T(diag): Time from diagnosis. T(onset): Time from onset. PPMS: primary progressive MS. nSPMS: non-active secondary progressive MS. N/A: not applicable.

The subpopulation defined as having ‘time from diagnosis to treatment initiation of greater than 2 years and time from onset to treatment initiation of greater than 5 years’ demonstrated a statistically significant difference (p<0.05) in change of EDSS from baseline following masitinib treatment as compared with placebo. This was true for patient categories of progressive MS, PPMS and non-active SPMS. Remarkably, all three categories also showed an overall reduction in disability as evidenced by reversal of the EDSS decline (i.e., negative change from baseline). Comparison to the full analysis dataset shows that the above referenced selected subpopulation had a 113% relative improvement for PPMS patients, a 64% relative improvement for progressive MS patients, and a 24% relative improvement for non-active SPMS patients.

In contrast, the complementary subpopulation defined as having ‘time from diagnosis to treatment initiation of no more than 2 years or time from onset to treatment initiation of no more than 5 years’ showed no significant masitinib treatment-effect with respect to placebo or even any discernable trend. Moreover, all patient categories show a continued increase in EDSS, corresponding to accumulating disability and disease progression, similar to (nonsignificant) or greater than placebo.

Without wishing to be bound by any theory, the Inventors suggest that the observed masitinib treatment-effect in the identified patient subpopulation is due to the parameters of ‘time from diagnosis’ and ‘time from onset’ correlating with dynamic pathophysiological influence of the drug's targeted mechanism; for example, evolving processes such as central nervous system (CNS)-compartmentalization. This model of MS considers that active forms of MS (RRMS and active SPMS) are predominantly driven by peripheral adaptive immunity (e.g., B cell and T cell lymphocytes), whereas progressive MS (PPMS and non-active SPMS) is predominantly driven by self-perpetuating innate immunity-related inflammation that has become contained within the CNS. Such a shift in disease pathophysiology is emphasized by the failure of RRMS therapies to alter disease progression in progressive MS patients. Hence, by targeting this progressive stage of MS, for example via modulation of activated macrophage/microglia and mast cells, it may be possible to inhibit compartmentalized neuroinflammation and promote CNS repair pathways.

### Example 3

Subgroup analyses based solely on ‘time from diagnosis to treatment initiation’ and solely on ‘time from clinical onset to treatment initiation’ are summarized below in Tables 6 to 8 and Tables 9 to 11, respectively. These data again show that orally administered masitinib (4.5 mg/kg/day) provides greater therapeutic benefit to a distinct subpopulation of the ‘progressive MS’ population. These data were also used to define cutoff values for the combinatory clinical marker ‘time from onset/diagnosis’ (see Example 2).

Analysis of Progressive MS Subpopulation According to the Clinical Marker ‘Time from Diagnosis to Treatment Initiation’ (Study AB07002)

In the absence of established prognostic or predictive biological markers that identify which progressive MS patients are most likely to benefit from masitinib treatment, it is necessary to use accessible clinical markers. The variable ‘time from diagnosis’ (at baseline or treatment initiation) has potential predictive value in showing whether it is beneficial to treat at an earlier or later stage of the disease process.

For patients with progressive MS (including both PPMS and non-active SPMS patients) the subpopulation with time from diagnosis to treatment initiation of greater than 2 years showed a significant masitinib treatment-effect with respect to placebo (ΔEDSS=−0.133, p=0.0033, corresponding to a 34% improvement in treatment-effect when compared with the full analysis dataset) and an overall reduction in disability for the masitinib treatment-arm (see Table 6 below). In contrast, the complement subpopulation of progressive MS patients with time from diagnosis to treatment initiation of no more than 2 year showed no discernable treatment-effect with respect to placebo.

TABLE 6
Comparison of masitinib- versus placebo-treated patients
in progressive MS subpopulations defined using the clinical
marker of time from diagnosis to treatment initiation

| PROG | N | | LSM EDSS | | | | |
|---|---|---|---|---|---|---|---|
| MS | MAS | PBO | MAS | PBO | ΔEDSS (CI) | P-value | R % |
| T(diag) > 1 Y | 178 | 92 | −0.012 | 0.097 | −0.109 | 0.0151 | 10% |
| | | | | | (−0.198, −0.021) | | |
| T(diag) ≤ 1 Y | 21 | 9 | −0.099 | −0.359 | 0.261 | | |
| | | | | | (0.016, 0.5055) | | |
| T(diag) > 2 Y | 165 | 82 | −0.029 | 0.104 | −0.133 | 0.0033 | 34% |
| | | | | | (−0.222, −0.044) | | |
| T(diag) ≤ 2 Y | 34 | 19 | 0.163 | 0.023 | 0.141 | | |
| | | | | | (−0.071, 0.3528) | | |
| T(diag) > 3 Y | 155 | 74 | −0.068 | 0.070 | −0.138 | 0.0013 | 39% |
| | | | | | (−0.222, −0.053) | | |
| T(diag) ≤ 3 Y | 44 | 27 | 0.056 | 0.004 | 0.053 | | |
| | | | | | (−0.167, 0.2721) | | |
| T(diag) > 4 Y | 137 | 62 | −0.053 | 0.094 | −0.147 | 0.0009 | 48% |
| | | | | | (−0.234, −0.060) | | |
| T(diag) ≤ 4 Y | 62 | 39 | 0.075 | 0.103 | −0.028 | | |
| | | | | | (−0.229, 0.1722) | | |

PROG MS: progressive MS. MAS: Masitinib. PBO: Placebo. LSM: Least squares means. EDSS: Expanded Disability Status Scale. ΔEDSS: Between group difference (treatment-effect). R %: relative difference in ΔEDSS with respect to full analysis dataset. T(diag): Time from diagnosis.

For patients with PPMS, a time from diagnosis to treatment initiation of greater than 2 years demarcated a subpopulation that first showed a strongly significant masitinib treatment-effect with respect to placebo (ΔEDSS=−0.246, p=0.0119, corresponding to an 85% improvement in treatment-effect when compared with the full analysis dataset) (see Table 7 below). An overall reduction in disability was also observed for the masitinib-arm of this subpopulation, as evidenced by reversal of the EDSS decline relative to baseline. In contrast, the complement subpopulation of PPMS patients with time from diagnosis to treatment initiation of no more than 2 year showed no discernable treatment-effect with respect to placebo.

For patients with PPMS, a time from diagnosis to treatment initiation of greater than 3 years also defined a subpopulation that showed a significant masitinib treatment-effect with respect to placebo (ΔEDSS=−0.211, p=0.0229, corresponding to an 59% improvement in treatment-effect when compared with the full analysis dataset) and an overall reduction in disability for the masitinib-arm, as evidenced by reversal of the EDSS decline relative to baseline (see Table 7 below).

TABLE 7
Comparison of masitinib- versus placebo-treated patients in PPMS subpopulations
defined using the clinical marker of time from diagnosis to treatment initiation

| | N | | LSM EDSS | | | | |
|---|---|---|---|---|---|---|---|
| PPMS | MAS | PBO | MAS | PBO | ΔEDSS (CI) | P-value | R % |
| T(diag) > 1 Y | 60 | 37 | −0.004 | 0.170 | −0.174 | 0.0445 | 31% |
| | | | | | (−0.344, −0.004) | | |
| T(diag) ≤ 1 Y | 19 | 8 | −0.097 | −0.234 | 0.137 | | |
| | | | | | (−0.104, 0.3786) | | |
| T(diag) > 2 Y | 48 | 27 | −0.070 | 0.176 | −0.246 | 0.0119 | 85% |
| | | | | | (−0.437, −0.054) | | |
| T(diag) ≤ 2 Y | 31 | 18 | 0.190 | 0.113 | 0.077 | | |
| | | | | | (−0.174, 0.3285) | | |
| T(diag) > 3 Y | 41 | 20 | −0.099 | 0.112 | −0.211 | 0.0229 | 59% |
| | | | | | (−0.393, −0.029) | | |
| T(diag) ≤ 3 Y | 38 | 25 | 0.070 | 0.060 | 0.009 | | |
| | | | | | (−0.242, 0.2607) | | |
| T(diag) > 4 Y | 32 | 11 | 0.097 | 0.263 | −0.166 | 0.0573 | 25% |
| | | | | | (−0.338, 0.0051) | | |
| T(diag) ≤ 4 Y | 47 | 34 | 0.099 | 0.188 | −0.089 | | |
| | | | | | (−0.340, 0.1625) | | |

PPMS: primary progressive multiple sclerosis. MAS: Masitinib. PBO: Placebo. LSM: Least squares means. EDSS: Expanded Disability Status Scale. ΔEDSS: Between group difference (treatment-effect). R %: relative difference in ΔEDSS with respect to full analysis dataset. T(diag): Time from diagnosis.

For patients with non-active SPMS the subpopulation with time from diagnosis to treatment initiation of greater than 2 years or greater than 3 years also showed a significant masitinib treatment-effect with respect to placebo and an overall reduction in disability for the masitinib treatment-arm (see Table 8 below). In contrast, the complement subpopulation of non-active SPMS patients with time from diagnosis to treatment initiation of no more than 2 year showed no discernable treatment-effect with respect to placebo.

TABLE 8
Comparison of masitinib- versus placebo-treated patients
in non-active SPMS subpopulations defined using the clinical
marker of time from diagnosis to treatment initiation

| | N | | LSM EDSS | | | | |
|---|---|---|---|---|---|---|---|
| nSPMS | MAS | PBO | MAS | PBO | ΔEDSS (CI) | P-value | R % |
| T(diag) > 1 Y | 118 | 55 | −0.039 | 0.062 | −0.101 | 0.0257 | 6% |
| | | | | | (−0.189, −0.012) | | |
| T(diag) ≤ 1 Y | 2 | 1 | N/A | N/A | N/A | | |
| T(diag) > 2 Y | 117 | 55 | −0.039 | 0.064 | −0.102 | 0.0241 | 7% |
| | | | | | (−0.192, −0.013) | | |
| T(diag) ≤ 2 Y | 3 | 1 | N/A | N/A | N/A | | |
| T(diag) > 3 Y | 114 | 54 | −0.045 | 0.062 | −0.107 | 0.0195 | 13% |
| | | | | | (−0.196, −0.017) | | |
| T(diag) ≤ 3 Y | 6 | 2 | N/A | N/A | N/A | | |
| T(diag) > 4 Y | 105 | 51 | −0.050 | 0.073 | −0.123 | 0.0094 | 29% |
| | | | | | (−0.216, −0.030) | | |
| T(diag) ≤ 4 Y | 15 | 5 | 0.036 | 0.041 | −0.004 | | |
| | | | | | (−0.119, 0.1098) | | |

nSPMS: non-active secondary progressive MS. MAS: Masitinib. PBO: Placebo. LSM: Least squares means. EDSS: Expanded Disability Status Scale. ΔEDSS: Between group difference (treatment-effect). R %: relative difference in ΔEDSS with respect to full analysis dataset. T(diag): Time from diagnosis. N/A: not applicable.

Analysis of Progressive MS Subpopulation According to the Clinical Marker ‘Time from Onset to Treatment Initiation’ (Study AB07002)

In the absence of established prognostic or predictive biological markers that identify which progressive MS patients are most likely to benefit from masitinib treatment, it is necessary to use accessible clinical markers. The variable ‘time from clinical onset’ has potential predictive value in showing whether it is beneficial to treat at an earlier or later stage of the disease process.

For patients with progressive MS (including both PPMS and non-active SPMS patients), the subpopulation defined by time from onset to treatment initiation of greater than 5 years showed a significant masitinib treatment-effect with respect to placebo (AEDSS=−0.156, p=0.0003, corresponding to a 58% improvement in treatment-effect when compared with the full analysis dataset) and an overall reduction in disability for the masitinib treatment-arm (see Table 9 below). In contrast, the complement subpopulation of progressive MS patients with time from onset to treatment initiation of no more than 5 years, showed no discernable treatment-effect with respect to placebo.

TABLE 9
Comparison of masitinib-versus placebo-treated patients
in progressive MS subpopulations defined using the clinical
marker of time from onset to treatment initiation

| PROG | N | | LSM EDSS | | | | |
|---|---|---|---|---|---|---|---|
| MS | MAS | PBO | MAS | PBO | ΔEDSS (CI) | P-value | R % |
| T(onset) > 1 Y | 198 | 101 | 0.012 | 0.106 | −0.094 | 0.0284 | −5% |
| | | | | | (−0.178, −0.009) | | |
| T(onset) ≤ 1 Y | 1 | 0 | N/A | N/A | N/A | | |
| T(onset) > 2 Y | 194 | 101 | 0.004 | 0.104 | −0.100 | 0.0208 | 1% |
| | | | | | (−0.184, −0.015) | | |
| T(onset) ≤ 2 Y | 5 | 0 | N/A | N/A | N/A | | |
| T(onset) > 3 Y | 188 | 91 | −0.010 | 0.109 | −0.119 | 0.0071 | 20% |
| | | | | | (−0.206, −0.032) | | |
| T(onset) ≤ 3 Y | 11 | 10 | 0.106 | −0.240 | 0.346 | | |
| | | | | | (−0.378, 1.0704) | | |
| T(onset) > 4 Y | 172 | 85 | −0.027 | 0.101 | −0.128 | 0.0048 | 29% |
| | | | | | (−0.217, −0.039) | | |
| T(onset) ≤ 4 Y | 27 | 16 | −0.000 | −0.146 | 0.146 | | |
| | | | | | (−0.055, 0.3469) | | |
| T(onset) > 5 Y | 162 | 82 | −0.031 | 0.125 | −0.156 | 0.0003 | 58% |
| | | | | | (−0.241, −0.070) | | |
| T(onset) ≤ 5 Y | 37 | 19 | 0.259 | −0.003 | 0.261 | | |
| | | | | | (0.048, 0.4753) | | |

PROG MS: progressive MS. MAS: Masitinib. PBO: Placebo. LSM: Least squares means. EDSS: Expanded Disability Status Scale. ΔEDSS: Between group difference (treatment-effect). R %: relative difference in ΔEDSS with respect to full analysis dataset. T(onset): Time from onset. N/A: not applicable.

For patients with PPMS, a time from onset to treatment initiation of greater than 5 years demarcated a subpopulation that showed a significant masitinib treatment-effect with respect to placebo (ΔEDSS=−0.214, p=0.0176, corresponding to a 61% improvement in treatment-effect when compared with the full analysis dataset) and an overall reduction in disability for the masitinib treatment-arm (see Table 10 below). In contrast, the complement subpopulation of PPMS patients with time from onset to treatment initiation of no more than 5 years showed no discernable treatment-effect with respect to placebo.

TABLE 10
Comparison of masitinib- versus placebo-treated patients in PPMS subpopulations
defined using the clinical marker of time from onset to treatment initiation

| | N | | LSM EDSS | | | | |
|---|---|---|---|---|---|---|---|
| PPMS | MAS | PBO | MAS | PBO | ΔEDSS (CI) | P-value | R % |
| T(onset) > 1 Y | 78 | 45 | 0.040 | 0.166 | −0.125 | 0.1214 | −6% |
| | | | | | (−0.284, 0.0332) | | |
| T(onset) ≤ 1 Y | 1 | 0 | N/A | N/A | N/A | | |
| T(onset) > 2 Y | 74 | 45 | 0.026 | 0.165 | −0.139 | 0.0861 | 5% |
| | | | | | (−0.297, 0.0197) | | |
| T(onset) ≤ 2 Y | 1 | 0 | N/A | N/A | N/A | | |
| T(onset) > 3 Y | 68 | 36 | 0.021 | 0.195 | −0.174 | 0.0598 | 31% |
| | | | | | (−0.356, 0.0071) | | |
| T(onset) ≤ 3 Y | 11 | 9 | 0.111 | −0.238 | 0.349 | | |
| | | | | | (−0.395, 1.0925) | | |
| T(onset) > 4 Y | 53 | 32 | 0.028 | 0.189 | −0.162 | 0.0991 | 22% |
| | | | | | (−0.354, 0.0304) | | |
| T(onset) ≤ 4 Y | 26 | 13 | −0.082 | −0.133 | 0.051 | | |
| | | | | | (−0.169, 0.2717) | | |
| T(onset) > 5 Y | 47 | 29 | 0.029 | 0.244 | −0.214 | 0.0176 | 61% |
| | | | | | (−0.391, −0.037) | | |
| T(onset) ≤ 5 Y | 32 | 16 | 0.314 | −0.026 | 0.340 | | |
| | | | | | (−0.004, 0.6835) | | |

PPMS: primary progressive multiple sclerosis. MAS: Masitinib. PBO: Placebo. LSM: Least squares means. EDSS: Expanded Disability Status Scale. ΔEDSS: Between group difference (treatment-effect). R %: relative difference in ΔEDSS with respect to full analysis dataset. T(onset): Time from onset. N/A: not applicable.

For patients with non-active SPMS, the subpopulation defined by time from onset to treatment initiation of greater than 5 years also showed a significant masitinib treatment-effect with respect to placebo and an overall reduction in disability for the masitinib treatment-arm (see Table 11 below). In contrast, the complement subpopulation of non-active SPMS patients with time from onset to treatment initiation of no more than 5 year showed no discernable treatment-effect with respect to placebo.

TABLE 11
Comparison of masitinib- versus placebo-treated patients
in non-active SPMS subpopulations defined using the clinical
marker of time from onset to treatment initiation

| | N | | LSM EDSS | | | | |
|---|---|---|---|---|---|---|---|
| nSPMS | MAS | PBO | MAS | PBO | ΔEDSS (CI) | P-value | R % |
| T(onset) > 1 Y | 120 | 56 | −0.035 | 0.059 | −0.095 | 0.0347 | 0% |
| | | | | | (−0.182, −0.006) | | |
| T(onset) ≤ 1 Y | 0 | 0 | N/A | N/A | N/A | | |
| T(onset) > 2 Y | 120 | 56 | −0.035 | 0.059 | −0.095 | 0.0347 | 0% |
| | | | | | (−0.182, −0.006) | | |
| T(onset) ≤ 2 Y | 0 | 0 | N/A | N/A | N/A | | |
| T(onset) > 3 Y | 120 | 55 | −0.037 | 0.065 | −0.102 | 0.0229 | 7% |
| | | | | | (−0.190, −0.014) | | |
| T(onset) ≤ 3 Y | 0 | 0 | N/A | N/A | N/A | | |
| T(onset) > 4 Y | 119 | 53 | −0.040 | 0.070 | −0.110 | 0.0132 | 16% |
| | | | | | (−0.198, −0.023) | | |
| T(onset) ≤ 4 Y | 1 | 3 | N/A | N/A | N/A | | |
| T(onset) > 5 Y | 115 | 53 | −0.050 | 0.067 | −0.117 | 0.0088 | 23% |
| | | | | | (−0.205, −0.029) | | |
| T(onset) ≤ 5 Y | 5 | 3 | N/A | N/A | N/A | | |

nSPMS: non-active secondary progressive MS. MAS: Masitinib. PBO: Placebo. LSM: Least squares means. EDSS: Expanded Disability Status Scale. ΔEDSS: Between group difference (treatment-effect). R %: relative difference in ΔEDSS with respect to full analysis dataset. T(onset): Time from onset. N/A: not applicable.

1.-14. (canceled)

15. A method for treating progressive multiple sclerosis (MS) in a patient in need thereof, comprising administering to the patient masitinib, or a pharmaceutically acceptable salt or solvate thereof, wherein said patient has a time from diagnosis to treatment initiation with masitinib, or the pharmaceutically acceptable salt or solvate thereof, greater than 2 years and/or has a time from onset to treatment initiation with masitinib, or the pharmaceutically acceptable salt or solvate thereof, greater than 5 years.

16. The method according to claim 15, wherein said patient has a time from diagnosis to treatment initiation with masitinib, or the pharmaceutically acceptable salt or solvate thereof, greater than 2 years.

17. The method according to claim 15, wherein said patient has a time from onset to treatment initiation with masitinib, or the pharmaceutically acceptable salt or solvate thereof, greater than 5 years.

18. The method according to claim 15, wherein said patient has a time from diagnosis to treatment initiation with masitinib, or the pharmaceutically acceptable salt or solvate thereof, greater than 2 years and has a time from onset to treatment initiation with masitinib, or the pharmaceutically acceptable salt or solvate thereof, greater than 5 years.

19. The method according to claim 15, wherein progressive MS is primary progressive multiple sclerosis (PPMS).

20. The method according to claim 15, wherein progressive MS is non-active secondary progressive multiple sclerosis (non-active SPMS).

21. The method according to claim 15, wherein the pharmaceutically acceptable salt of masitinib is masitinib mesilate.

22. The method according to claim 15, wherein said masitinib, or the pharmaceutically acceptable salt or solvate thereof, is administered orally.

23. The method according to claim 15, wherein said masitinib, or the pharmaceutically acceptable salt or solvate thereof, is administered at a dose ranging from about 1 mg/kg/day to about 12 mg/kg/day.

24. The method according to claim 15, wherein said masitinib, or the pharmaceutically acceptable salt or solvate thereof, is administered at a dose of about 4.5 mg/kg/day.

25. The method according to claim 15, wherein said masitinib, or the pharmaceutically acceptable salt or solvate thereof, is administered at an initial dose of about 4.5 mg/kg/day during at least 12 weeks, and then at a dose of about 6 mg/kg/day thereafter, with each dose escalation being subjected to toxicity controls.

26. The method according to claim 15, wherein said masitinib, or the pharmaceutically acceptable salt or solvate thereof, is administered as a second-line treatment.

27. The method according to claim 15, wherein said patient failed to respond to a previously administered first-line treatment for multiple sclerosis or showed irreversible progression of the disease despite previous administration of a first-line treatment for multiple sclerosis.

28. The method according to claim 15, wherein said masitinib, or the pharmaceutically acceptable salt or solvate thereof, is administered with at least one further pharmaceutically active agent.

29. The method according to claim 28, wherein the at least one further pharmaceutically active agent is selected from the group consisting of adrenocorticotropic hormone (ACTH), alemtuzumab, azathioprine, high-dose biotin (MD1003), cladribine, cyclophosphamide, cyclosporine, dalfampridine, dimethyl fumarate, diroximel fumarate, evobrutinib, fampridine, fingolimod, glatiramer acetate, ibudilast, immunoglobulins, interferon, laquinimod, methotrexate, mitoxantrone, mycophenolate mofetil, natalizumab, ocrelizumab, ofatumumab, ozanimod, ponesimod, simvastatin, siponimod, teriflunomide, and ublituximab.

30. The method according to claim 29, wherein interferon is selected from interferon beta 1b, interferon beta-1a, and peginterferon beta-1a.
